(19) United States
(12) Patent Application Publication
(10) Pub. No.: US 2022/0118117 A1
(43) Pub. Date: Apr. 21, 2022

(54) MODULATORS OF METABOTROPIC GLUTAMATE RECEPTOR 4

(71) Applicant: The General Hospital Corporation, Boston, MA (US)

(72) Inventors: Zhaoda Zhang, Methuen, MA (US); Junfeng Wang, Malden, MA (US); Timothy M. Shoup, Franklin, MA (US); Anna-Liisa Brownell, Salem, MA (US)

(21) Appl. No.: 17/421,239

(22) PCT Filed: Jan. 8, 2020
(86) PCT No.: PCT/US20/12765

§ 371 Date: Jul. 7, 2021

Related U.S. Application Data
(60) Provisional application No. 62/789,562, filed on Jan. 8, 2019.

(51) Int. Cl. A61K 51/04; C07D 213/81; C07B 59/00

(52) U.S. Cl. CPC A61K 51/0455; C07B 2200/05; C07B 59/002; C07D 213/81

(57) ABSTRACT

The present application provides picolinamide compounds that can be used as allosteric positron emission tomography (“PET”) imaging probes. Methods of using these compounds for treating a neurodegenerative disease are also provided.

### CLAIM OF PRIORITY

This application claims priority to U.S. Provisional Patent Application Ser. No. 62/789,562, filed on Jan. 8, 2019, the entire contents of which are hereby incorporated by reference.

### FEDERALLY SPONSORED RESEARCH AND DEVELOPMENT

This invention was made with government support under Grant Nos. 1R01EB021708 and R01NS100164 awarded by the National Institutes of Health (NIH). The government has certain rights in the invention.

### TECHNICAL FIELD

This invention relates to metabotropic glutamate receptor 4 (“mGluR4”) positive allosteric modulators, and more particularly, to picolinamide derivatives that can be used as allosteric positron emission tomography (“PET”) imaging probes.

### BACKGROUND

There are numerous deadly diseases affecting current human population. For example, neurodegenerative diseases affect a significant segment of population, especially the elderly. Parkinson's disease (“PD”), a progressive nervous system disorder that affects movement, affects more than 10 million people worldwide with an estimated total annual economic burden of more than $52 billion. PD was first described about 200 years ago but there is still no cure for this debilitating disease, only alleviate approaches for the symptoms.

### SUMMARY

The compounds of the present disclosure are mGluR4 positive allosteric modulators and can be used, for example, as ligands for the PET imaging of mGluR4 in the brain. The exemplary compounds display central nervous system (“CNS”) drug-like properties, including mGluR4 affinity, potent mGluR4 positive allosteric modulator (“PAM”) activity and selectivity against other mGluRs, as well as sufficient metabolic stability. Ex vivo biodistribution studies showed reversible binding of ¹⁸F labeled compounds in all investigated tissues including the brain, liver, heart, lungs, and kidneys. PET imaging studies in male Sprague-Dawley rats showed that exemplary ¹⁸F labeled compounds accumulate in the brain regions known to express mGluR4. Pretreatment with the corresponding unlabeled compounds (¹⁹F compounds) as well as the other mGluR4 allosteric ligands, followed by imaging the brain with the ¹⁸F-labeled compounds, showed significant dose-dependent blocking effects on the receptor, which indicate that exemplary ¹⁸F labeled compounds bind specifically to mGluR4. These results show that the unlabeled compounds of the present disclosure are useful for treating or ameliorating symptoms of diseases or conditions in which mGluR4 is implicated (e.g., PD).

In some embodiments, the present disclosure provides a compound of Formula (I):

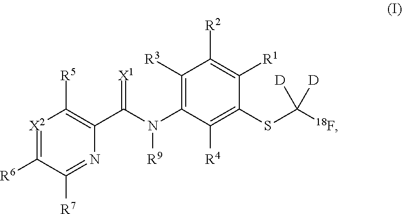

or a pharmaceutically acceptable salt thereof, wherein:

R¹ is selected from halo, CN, NO₂, C₁₋₆ alkylthio, C₁₋₃ haloalkyl, C₁₋₃ haloalkoxy, 4-10 membered heterocycloalkyl, NHC(O)Cy¹, NHS(O)₂Cy¹, C(O)NHCy¹, and S(O)₂NHCy¹; wherein said 4-10 membered heterocycloalkyl is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy;

each Cy¹ is independently an C₆₋₁₀ aryl, optionally substituted 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy;

R², R³, and R⁴ are each independently selected from H, OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, amino, C₁₋₆ alkylamino, di(C₁₋₆ alkyl)amino, thio, and C₁₋₆ alkylthio;

R⁹ is selected from H and C₁₋₃ alkyl;

X² is selected from N and CR⁸;

R⁵, R⁶, R⁷, and R⁸ are each independently selected from H, OH, NO₂, CN, halo, C₁₋₃ alkyl, C₁₋₃ alkoxy, C₁₋₃ haloalkyl, C₁₋₃ haloalkoxy, cyano-C₁₋₃ alkyl, HO—C₁₋₃ alkyl, amino, C₁₋₆ alkylamino, di(C₁₋₆ alkyl)amino, thio, and C₁₋₆ alkylthio;

X¹ is selected from O, S, and NR^{e1}; and

R^{e1} is selected from H, C₁₋₄ alkyl, C₁₋₄ alkoxy, OH, and CN;

or R⁵ and R^{e1} together with the N atom to which R^{e1} is attached and the carbon atom to which R⁵ is attached for a 5-10 membered heterocycloalkyl ring, which is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy.

In some embodiments:

R¹ is selected from halo, NO₂, C₁₋₆ alkylthio, 4-10 membered heterocycloalkyl, NHC(O)Cy¹, and S(O)₂NHCy¹; wherein said 4-10 membered heterocycloalkyl is optionally substituted with halo or C₁₋₃ alkyl;
each Cy¹ is independently C₆₋₁₀ aryl, optionally substituted with halo or C₁₋₃ alkyl;

R², R³, and R⁴ are each independently selected from H, NO₂, halo, C₁₋₃ alkyl, C₁₋₃ alkoxy, C₁₋₃ alkylthio, and C₁₋₃ haloalkyl;

R⁹ is H; and

R⁵, R⁶, R⁷, and R⁸ are each independently selected from H, OH, amino, halo, C₁₋₃ alkyl, C₁₋₃ alkoxy, C₁₋₃ haloalkyl, thio, and C₁₋₆ alkylthio.

In some embodiments, the compound of Formula (I) has formula:

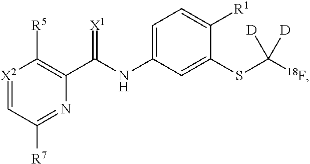

or a pharmaceutically acceptable salt thereof, wherein:

R¹ is selected from halo and NO₂;

X¹ is selected from O and S;

X² is selected from N and CH;

R⁵ is selected from H, amino, and OH; and

R⁷ is selected from H and halo.

In some embodiments, the compound has formula:

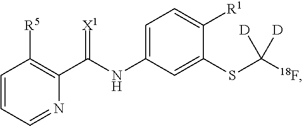

or a pharmaceutically acceptable salt thereof, wherein:

R⁵ is selected from H and amino.

In some embodiments, the compound of Formula (I) has formula:

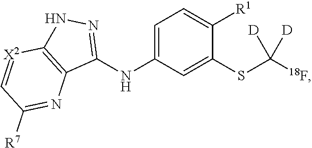

or a pharmaceutically acceptable salt thereof, wherein:

R¹ is selected from halo and NO₂;

X² is selected from N and CH; and

R⁷ is selected from H and halo.

In some embodiments, the compound of Formula (I) is selected from any one of the following compounds, or a pharmaceutically acceptable salt thereof:

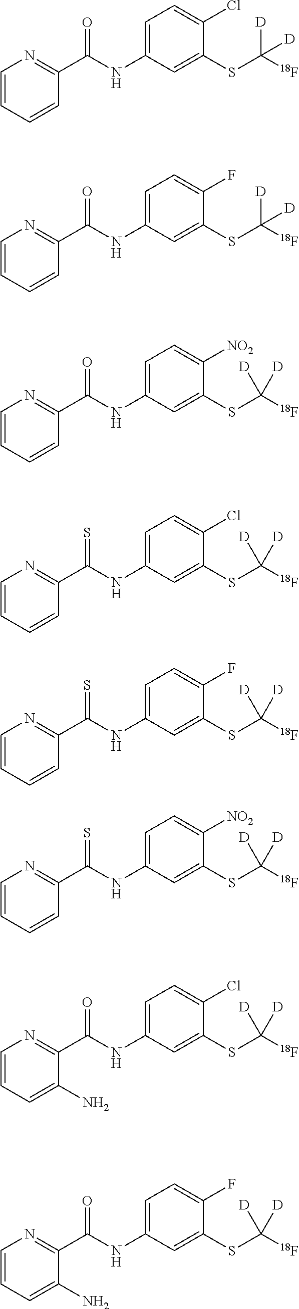

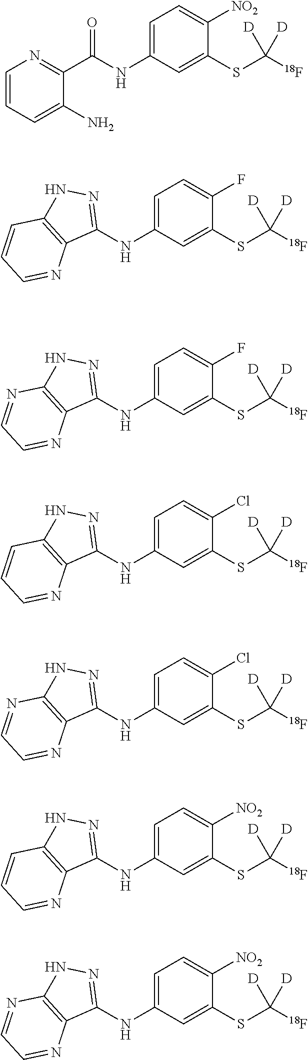

In some embodiments, the present disclosure provides a pharmaceutical composition comprising a compound of Formula (I), or a pharmaceutically acceptable salt thereof, and a pharmaceutically acceptable carrier.

In some embodiments, the present disclosure provides a method of imaging a brain of a subject, the method comprising:

i) administering to the subject an effective amount of a compound of Formula (I), or a pharmaceutically acceptable salt thereof;

ii) waiting a time sufficient to allow the compound of Formula (I) to accumulate in the brain to be imaged; and

iii) imaging the brain with an imaging technique.

In some embodiments, the present disclosure provides a method of monitoring treatment of a neurological disorder associated with mGluR4 in a subject, the method comprising:

i) administering to the subject an effective amount of a compound of Formula (I), or a pharmaceutically acceptable salt thereof;

ii) waiting a time sufficient to allow the compound of Formula (I) to accumulate in a brain of the subject;

iii) imaging the brain of the subject with an imaging technique;

iv) administering to the subject a therapeutic agent in an effective amount to treat the neurological disorder;

v) after iv), administering to the subject an effective amount of a compound of Formula (I), or a pharmaceutically acceptable salt thereof;

vi) waiting a time sufficient to allow the compound of Formula (I) to accumulate in the brain of the subject;

vii) imaging the brain of the subject with an imaging technique; and

viii) comparing the image of step iii) and the image of step vii).

In some embodiments, the imaging technique is selected from positron emission tomography (PET) imaging, positron emission tomography with computer tomography (PET/CT) imaging, and positron emission tomography with magnetic resonance (PET/MRI) imaging.

In some embodiments, the neurological disorder associated with mGluR4 is selected from Parkinson's disease, dyskinesia, Lewy body disease, Prion disease, motor neuron disease (MND), and Huntington's disease.

In some embodiments, the present disclosure provides a compound of Formula (II):

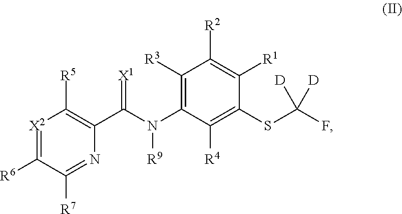

or a pharmaceutically acceptable salt thereof, wherein:

R¹ is selected from halo, CN, NO₂, C₁₋₆ alkylthio, C₁₋₃ haloalkyl, C₁₋₃ haloalkoxy, 4-10 membered heterocycloalkyl, NHC(O)Cy¹, NHS(O)₂Cy¹, C(O)NHCy¹, and S(O)₂NHCy¹; wherein said 4-10 membered heterocycloalkyl is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy;

each Cy¹ is independently an C₆₋₁₀ aryl, optionally substituted 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy;

R², R³, and R⁴ are each independently selected from H, OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, amino, C₁₋₆ alkylamino, di(C₁₋₆ alkyl)amino, thio, and C₁₋₆ alkylthio;

R⁹ is selected from H and C₁₋₃ alkyl;

X² is selected from N and CR⁸;

R⁵, R⁶, R⁷, and R⁸ are each independently selected from H, OH, NO₂, CN, halo, C₁₋₃ alkyl, C₁₋₃ alkoxy, C₁₋₃ haloalkyl, C₁₋₃ haloalkoxy, cyano-C₁₋₃ alkyl, HO—C₁₋₃ alkyl, amino, C₁₋₆ alkylamino, di(C₁₋₆ alkyl)amino, thio, and C₁₋₆ alkylthio;

X¹ is selected from O, S, and NR^{e1}; and

R^{e1} is selected from H, C₁₋₄ alkyl, C₁₋₄ alkoxy, OH, and CN;

or R⁵ and R^{e1} together with the N atom to which R^{e1} is attached and the carbon atom to which R⁵ is attached for a 5-10 membered heterocycloalkyl ring, which is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy.

In some embodiments:

R¹ is selected from halo, NO₂, C₁₋₆ alkylthio, 4-10 membered heterocycloalkyl, NHC(O)Cy¹, and S(O)₂NHCy¹; wherein said 4-10 membered heterocycloalkyl is optionally substituted with halo or C₁₋₃ alkyl;

each Cy¹ is independently C₆₋₁₀ aryl, optionally substituted with halo or C₁₋₃ alkyl;

R², R³, and R⁴ are each independently selected from H, NO₂, halo, C₁₋₃ alkyl, C₁₋₃ alkoxy, C₁₋₃ alkylthio, and C₁₋₃ haloalkyl;

R⁹ is H; and

R⁵, R⁶, R⁷, and R⁸ are each independently selected from H, OH, amino, halo, C₁₋₃ alkyl, C₁₋₃ alkoxy, C₁₋₃ haloalkyl, thio, and C₁₋₆ alkylthio.

In some embodiments, the compound of Formula (II) has formula:

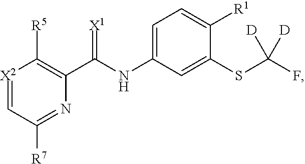

or a pharmaceutically acceptable salt thereof, wherein:

R¹ is selected from halo and NO₂;

X¹ is selected from O and S;

X² is selected from N and CH;

R⁵ is selected from H, amino, and OH; and

R⁷ is selected from H and halo.

In some embodiments, the compound has formula:

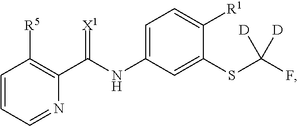

or a pharmaceutically acceptable salt thereof, wherein:

R⁵ is selected from H and amino.

In some embodiments, the compound of Formula (II) has formula:

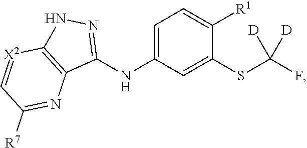

or a pharmaceutically acceptable salt thereof, wherein:

R¹ is selected from halo and NO₂;

X² is selected from N and CH; and

R⁷ is selected from H and halo.

In some embodiments, the compound of Formula (II) is selected from any one of the following compounds, or a pharmaceutically acceptable salt thereof:

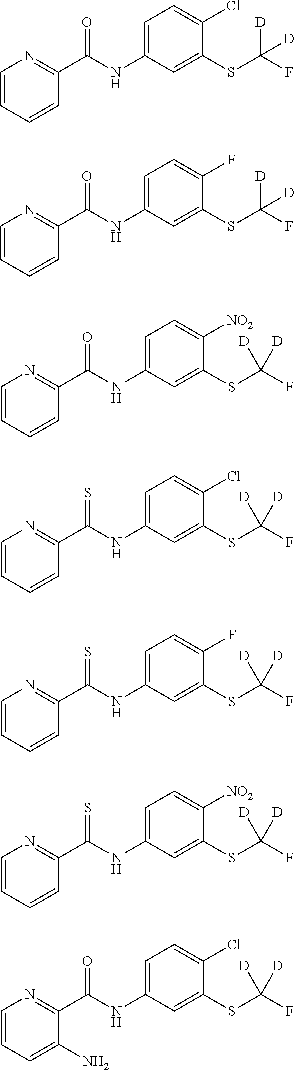

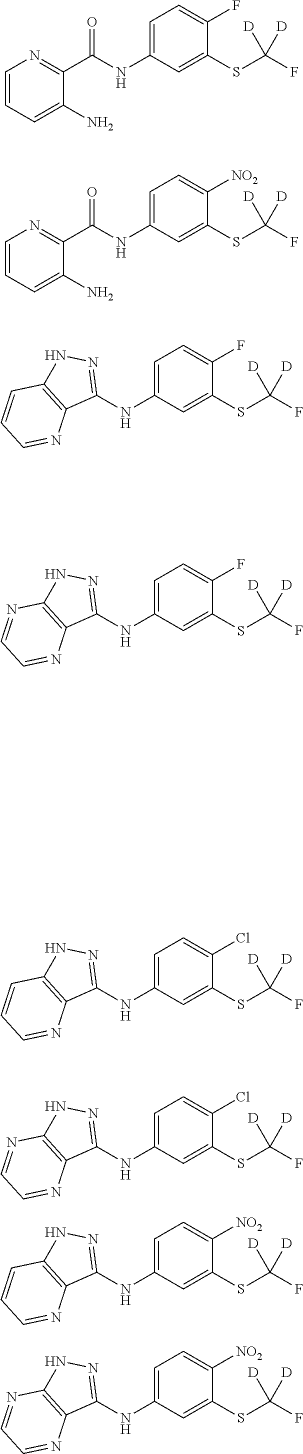

In some embodiments, the present disclosure provides pharmaceutical composition comprising a compound of Formula (II), or a pharmaceutically acceptable salt thereof, and a pharmaceutically acceptable carrier.

In some embodiments, the present disclosure provides a method of treating a neurological disorder associated with mGluR4 in a subject, the method comprising administering to the subject in need thereof a therapeutically effective amount of a compound of Formula (II), or a pharmaceutically acceptable salt thereof.

In some embodiments, the neurological disorder associated with mGluR4 is selected from Parkinson's disease, dyskinesia, Lewy body disease, Prion disease, motor neuron disease (MND), and Huntington's disease.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 contains chemical structures of representative mGluR4 PAMs

FIG. 2 contains chemical structures of PET tracers for mGluR4.

FIG. 3 contains a synthetic scheme showing synthesis of compound 15.

FIG. 4 contains a synthetic scheme showing synthesis of compound 18.

FIG. 5 contains a line plot showing the mGluR4 PAM activity.

FIG. 6 contains a table showing affinity, mGluR4 PAM activity and selectivity of compound 15.

FIG. 7 contains a line plot showing microsomal stability of compound 4.

FIG. 8 contains a line plot showing microsomal stability of compound 13.

FIG. 9 contains a line plot showing microsomal stability of compound 15.

FIG. 10 contains a line plot showing microsomal stability of propranolol.

FIG. 11 contains a line plot showing solution stability of compound 4.

FIG. 12 contains a line plot showing solution stability of compound 13.

FIG. 13 contains a line plot showing solution stability of compound 15.

FIG. 14 contains a line plot showing solution stability of Diltiazem.

FIG. 15 contains a line plot showing metabolic stability of compound 15.

FIG. 16 contains a bar graph showing biodistribution of [¹⁸F]15.

FIG. 17 contains an image showing distribution of [¹⁸F]15 in the brain.

FIG. 18 contains time-activity curves showing accumulation and washout in all investigated brain areas for compound [¹⁸F]15.

FIG. 19 contains a bar graph showing results of a blocking experiment of [¹⁸F]15 using “cold” compound 13.

FIG. 20 contains a bar graph showing that both “cold” 13 and 15 inhibit [¹⁸F]15 binding dose dependently.

FIG. 21 contains PET imaging of male Sprague Dawley rants using [¹⁸F]15.

### DETAILED DESCRIPTION

L-Glutamate is the most abundant excitatory neurotransmitter in the central nervous system (CNS) of vertebrates and mediates more than 50% of all synapses. Two major classes of receptors, ionotropic glutamate receptors (iGluRs) and metabotropic glutamate receptors (mGluRs), as well as transporters are involved in glutamate signaling. iGluRs, including the N-methyl-d-aspartate (NMDA), α-amino-3-hydroxy-5-methyl-4-isoxazolepropionate (AMPA), and kainite receptors are ligand-gated ion channels that mediate fast synaptic transmission. The mGluRs modulate the presynaptic glutamate release and/or postsynaptic effects of glutamate. mGluRs belong to class C of the G-protein-coupled receptor (GPCR) super family, which can be further divided into three subgroups including eight known receptor sub-types (group I: mGluR1 and mGluR5, group II: mGluR2 and mGluR3, and group III: mGluR4, mGluR6, mGluR7, and mGluR8) based on their structural similarity, ligand specificity, and preferred coupling mechanism. mGluRs are involved in glutamate signaling in excitatory synapses in CNS, and they have distinctive biodistribution in CNS depending on subtypes and subgroups.

mGluR4 is expressed at multiple synapses throughout the basal ganglia, mainly localized presynaptically and expressed in the striatum, hippocampus, thalamus, and cerebellum. mGluR4 participates in presynaptic neurotransmission of excitatory glutamate signaling and is implicated for various neuronal diseases such as Parkinson's disease (PD) and related disorders. As a group III mGluR, mGluR4 interacts with the G_{ai/o} subunit of G-protein that negatively couples with adenylate cyclase to inhibit cAMP dependent signaling pathways. Since mGluR4 receptors are localized on presynaptic site they are important contributors to glutamate neurotransmission and their activation reduces neurotransmitter release, a mechanism implicated in the pathophysiology of some neurodegenerative diseases such as the PD.

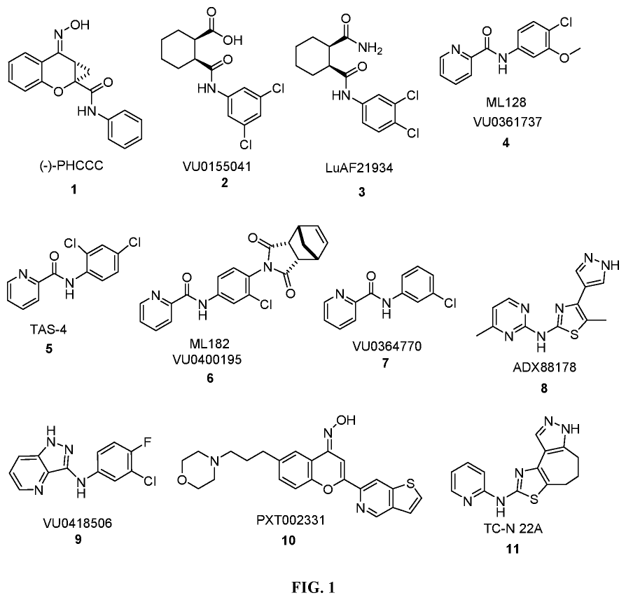

As a family C GPCR, the activation of mGluR4 can be accomplished or enhanced by two different mechanisms: orthosteric agonists or positive allosteric modulators (PAMs). Although most orthosteric ligands lack clear subtype-selectivity and/or blood-brain barrier (BBB) penetration, few examples of selective and brain penetrant orthosteric agonists exist, such as LSP4-2022. Allosteric modulators are small molecules capable of enhancing agonist or antagonist mediated receptor activity while possessing no or less intrinsic agonist or antagonist activity. Relative to classical mGluR agonists and antagonists, the positive allosteric modulators (PAMs) and negative allosteric modulators (NAMs) possess enhanced selectivity versus other mGluRs. Allosteric modulators also offer other benefits such as subtype-selectivity, retained physiology of the receptor and the saturation effects. Targeting mGluR4 with allosteric modulators offers enhanced therapeutic effects and improved side-effect profiles. One mGluR4 PAM, foliglurax (CAS Registry No. 1883329-53-0), is currently in phase II clinical trial for treating PD and dyskinesia associated with PD (NCT03331848). In addition, several mGluR4 PAMs have demonstrated antiparkinsonian activity in animal models of PD (see FIG. 1 and references 18-29).

Hence, mGluR4 modulators can be used for treating neurological and psychiatric disorders. In addition, allosteric modulators are useful as PET tracers for imaging mGluR4 and can provide insights into biological process at molecular level in vivo. PET has become an important clinical diagnostic and research modality, and also a valuable technology in drug discovery and development. PET offers picomolar sensitivity and is a fully translational technique that requires specific probes radiolabeled with a usually short-lived positron-emitting radionuclide. Carbon-11 (radioactive half-life (t_{1/2})=20.4 min) and fluorine-18 (t_{1/2}=109.7 min) are the most commonly used radionuclides in PET imaging. PET has provided the capability of measuring biological processes at the molecular and metabolic levels in vivo by the detection of the photons formed as a result of the annihilation of the emitted positrons. As a noninvasive medical and molecular imaging technique and a powerful tool in neurological research, PET offers the possibility of visualizing and analyzing the target receptor expression under physiological and pathophysiological conditions. PET can be used to detect disease-related biochemical changes before the disease-associated anatomical changes can be found using standard medical imaging modalities.

Moreover, PET tracers can be used as biomarkers during the clinical development of potential therapeutics, in which the receptor occupancy of potential drug candidates in the brain is measured. Knowledge of in vivo receptor occupancy answers many vital questions in the drug development process, such as whether potential drugs reach their molecular targets, the relationship between therapeutic dose and receptor occupancy, the correlation between receptor occupancy and plasma drug levels, and the duration of time the drug remains at its target.

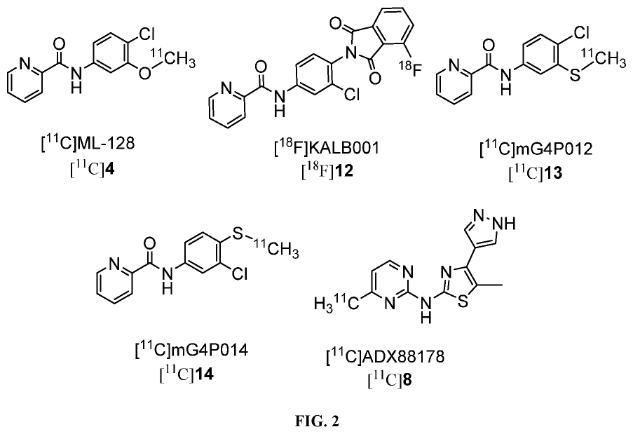

Despite the great wealth of information that such probes can provide, the potential of PET strongly depends on the availability of suitable PET radiotracers. While several mGluR4 PET tracers have been developed (see FIG. 2), these tracers suffer from distinct disadvantages, such as insufficient metabolic and chemical stability, fast wash-out, poor contrast, and half-life that is too short for imaging experiments.

The present disclosure provides, inter alia, picolinamide derivatives GluR4-selective positive allosteric modulators (PAMs) of the mGluR4. As described herein, these compounds show high affinity to mGluR4 as well as good selectivity relative to other mGlu receptors (e.g., mGlu 1, 5, 2, 3, 6, 7, or 8 receptors). Among other things, these compounds have enhanced metabolic stability. When labeled with ¹⁸F, these compounds can be advantageously used as mGluR4 tracers with superior imaging efficiency, with half-life that is long enough to carry out relatively extended imaging protocols. This facilitates kinetic studies and high-quality metabolic and plasma analysis.

Therapeutic Compounds

In some embodiments, the present disclosure provides a compound of Formula:

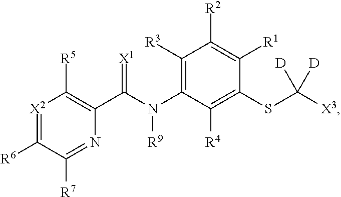

or a pharmaceutically acceptable salt thereof, wherein X³ is selected from ¹⁸F and F, and X¹, X², R¹, R², R³, R⁴, R⁵, R⁶, R⁷ and R⁹ are as described herein for Formula (I) or Formula (II).

In some embodiments, the present disclosure provides a compound of Formula (I):

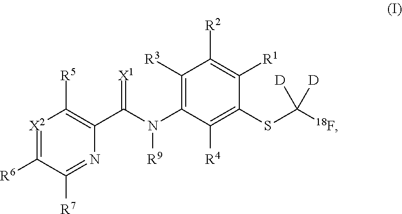

or a pharmaceutically acceptable salt thereof, wherein X¹, X², R¹, R², R³, R⁴, R⁵, R⁶, R⁷, and R⁹ are as described herein.

In some embodiments, the present disclosure provides a compound of Formula (II):

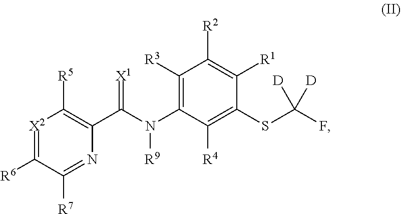

or a pharmaceutically acceptable salt thereof, wherein X¹, X², R¹, R², R³, R⁴, R⁵, R⁶, R⁷, and R⁹ are as described herein.

Certain embodiments of X¹, X², R¹, R², R³, R⁴, R⁵, R⁶, R⁷, and R⁹ are described below. In some embodiments:

R¹ is selected from halo, CN, NO₂, C₁₋₆ alkylthio, C₁₋₃ haloalkyl, C₁₋₃ haloalkoxy, 4-10 membered heterocycloalkyl, NHC(O)Cy¹, NHS(O)₂Cy¹, C(O)NHCy¹, and S(O)₂NHCy¹; wherein said 4-10 membered heterocycloalkyl is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy;

each Cy¹ is independently an C₆₋₁₀ aryl, optionally substituted 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy;

R², R³, and R⁴ are each independently selected from H, OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, amino, C₁₋₆ alkylamino, di(C₁₋₆ alkyl)amino, thio, and C₁₋₆ alkylthio;

R⁹ is selected from H and C₁₋₃ alkyl;

X² is selected from N and CR⁸;

R⁵, R⁶, R⁷, and R⁸ are each independently selected from H, OH, NO₂, CN, halo, C₁₋₃ alkyl, C₁₋₃ alkoxy, C₁₋₃ haloalkyl, C₁₋₃ haloalkoxy, cyano-C₁₋₃ alkyl, HO—C₁₋₃ alkyl, amino, C₁₋₆ alkylamino, di(C₁₋₆ alkyl)amino, thio, and C₁₋₆ alkylthio;

X¹ is selected from O, S, and NR^{e1}; and

R^{e1} is selected from H, C₁₋₄ alkyl, C₁₋₄ alkoxy, OH, and CN;

or R⁵ and R^{e1} together with the N atom to which R^{e1} is attached and the carbon atom to which R⁵ is attached for a 5-10 membered heterocycloalkyl ring, which is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy.

In some embodiments, R¹ is selected from halo, CN, NO₂, C₁₋₆ alkylthio, C₁₋₃ haloalkyl, C₁₋₃ haloalkoxy, 4-10 membered heterocycloalkyl, NHC(O)Cy¹, NHS(O)₂Cy¹, C(O)NHCy¹, and S(O)₂NHCy¹; wherein said 4-10 membered heterocycloalkyl is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy;

In some embodiments, R¹ is selected from halo, NO₂, C₁₋₆ alkylthio, 4-10 membered heterocycloalkyl, NHC(O)Cy¹, and S(O)₂NHCy¹; wherein said 4-10 membered heterocycloalkyl is optionally substituted with halo or C₁₋₃ alkyl.

R¹ is selected from halo and NO₂.

In some embodiments, R¹ is halo. In some embodiments, R¹ is Cl. In some embodiments, R¹ is F.

In some embodiments, R¹ is NO₂.

In some embodiments, R¹ is C₁₋₆ alkylthio.

In some embodiments, R¹ is 4-10 membered heterocycloalkyl, optionally substituted with halo or C₁₋₃ alkyl. In some embodiments, the 4-10 membered heterocycloalkyl is selected from succinimidyl, succinimidyl that is fused with a benzene ring or a cyclohexyl ring, and isothiazolidine. In some embodiments, the 4-10 membered heterocycloalkyl is substituted with 1 or 2 C₁₋₃ alkyl. In some embodiments, the 4-10 membered heterocycloalkyl is substituted with halo (e.g., Cl).

In some embodiments, R¹ is C₁₋₃ haloalkyl.

In some embodiments, R¹ is C₁₋₃ haloalkoxy.

In some embodiments, R¹ is NHC(O)Cy¹.

In some embodiments, R¹ is NHS(O)₂Cy¹.

In some embodiments, R¹ is C(O)NHCy¹.

In some embodiments, R¹ is S(O)₂NHCy¹.

In some embodiments, each Cy¹ is independently C₆₋₁₀ aryl, optionally substituted with halo or C₁₋₃ alkyl. In some embodiments, each Cy¹ is independently phenyl, optionally substituted with halo (e.g., Cl or F).

In some embodiments, R², R³, and R⁴ are each independently selected from H, NO₂, halo, C₁₋₃ alkyl, C₁₋₃ alkoxy, C₁₋₃ alkylthio, and C₁₋₃ haloalkyl.

In some embodiments, R², R³, and R⁴ are each independently selected from H, halo, C₁₋₃ alkyl, C₁₋₃ alkylthio, and C₁₋₃ haloalkyl. In some embodiments, R², R³, and R⁴ are each independently selected from H, halo, and C₁₋₃ alkyl. In some embodiments, R², R³, and R⁴ are each H.

In some embodiments, R⁹ is H. In some embodiments, R⁹ is C₁₋₃ alkyl.

In some embodiments, X² is N. In some embodiments, X² is CR⁸. In some embodiments, X² is selected from N and CH.

In some embodiments, R⁵, R⁶, R⁷, and R⁸ are each independently selected from H, OH, halo, C₁₋₃ alkyl, C₁₋₃ alkoxy, C₁₋₃ haloalkyl, C₁₋₃ haloalkoxy, amino, C₁₋₆ alkylamino, di(C₁₋₆ alkyl)amino, thio, and C₁₋₆ alkylthio. In some embodiments, R⁵, R⁶, R⁷, and R⁸ are each independently selected from H, OH, amino, halo, C₁₋₃ alkyl, C₁₋₃ alkoxy, C₁₋₃ haloalkyl, thio, and C₁₋₆ alkylthio.

In some embodiments, R⁶ is H; R⁵ is selected from H, amino, and OH; and R⁷ is selected from H and halo. In some embodiments, R⁵ is selected from H and amino. In some embodiments, R⁵ is amino. In some embodiments, R⁵, R⁶, R⁷, and R⁸ are each H. In some embodiments, R⁵ is amino, X² is N, and R⁶ and R⁷ are each H. In some embodiments, R⁵ is amino and R⁶, R⁷, and R⁸ are each H.

In some embodiments, X¹ is selected from O and S.

In some embodiments, X¹ is O.

In some embodiments, X¹ is S.

In some embodiments, X¹ is NR^{e1}.

In some embodiments, R^{e1} is H. In some embodiments, R^{e1} is C₁₋₄ alkyl. In some embodiments, R^{e1} is OH. In some embodiments, Rₑ₁ is CN.

In some embodiments, X¹ is NR^{e1} and R⁵ and R^{e1} together with the N atom to which R^{e1} is attached and the carbon atom to which R⁵ is attached for a 5-10 membered heterocycloalkyl ring, which is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy. In some embodiments, R⁵ and R^{e1} together form a pyrazolyl ring.

In some embodiments:

R¹ is selected from halo and NO₂;

X¹ is selected from O and S;

X² is selected from N and CH;

R⁵ is selected from H, amino, and OH; and

R⁷ is selected from H and halo.

In some embodiments:

R¹ is selected from halo and NO₂;

X² is selected from N and CH; and

R⁷ is selected from H and halo.

In some embodiments, the compound of Formula (I) has formula:

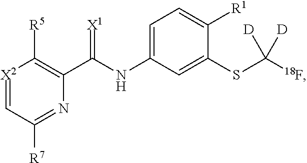

or a pharmaceutically acceptable salt thereof.

In some embodiments, the compound of Formula (I) has formula:

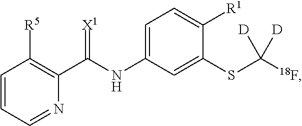

or a pharmaceutically acceptable salt thereof.

In some embodiments, the compound of Formula (I) has formula:

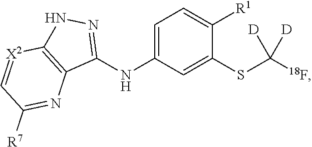

or a pharmaceutically acceptable salt thereof.

In some embodiments, the compound of Formula (I) is selected from any one of the following compounds, or a pharmaceutically acceptable salt thereof:

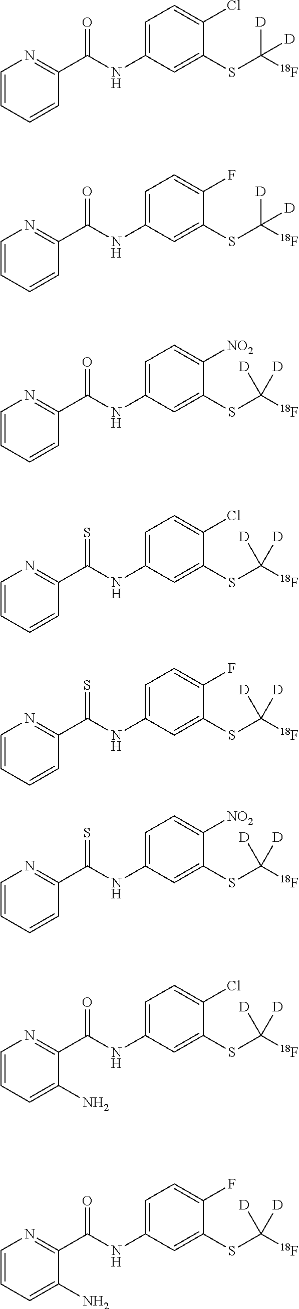

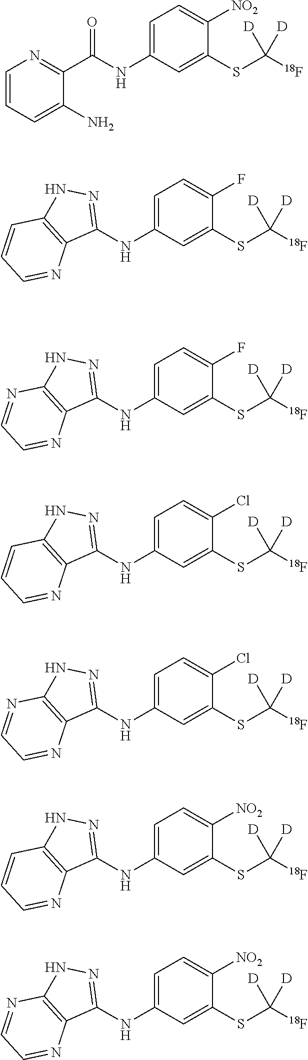

In some embodiments, the compound of Formula (II) has formula:

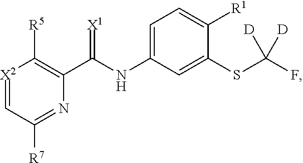

or a pharmaceutically acceptable salt thereof.

In some embodiments, the compound of Formula (II) has formula:

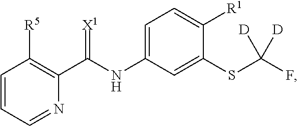

or a pharmaceutically acceptable salt thereof.

In some embodiments, the compound of Formula (II) has formula:

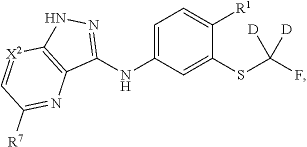

or a pharmaceutically acceptable salt thereof.

In some embodiments, the compound of Formula (II) is selected from any one of the following compounds, or a pharmaceutically acceptable salt thereof:

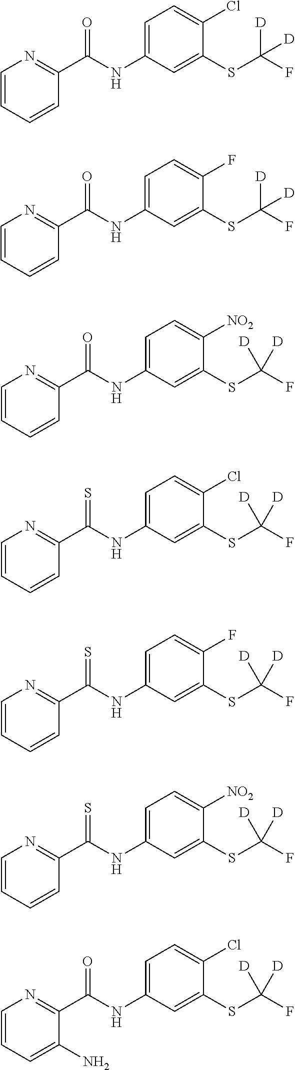

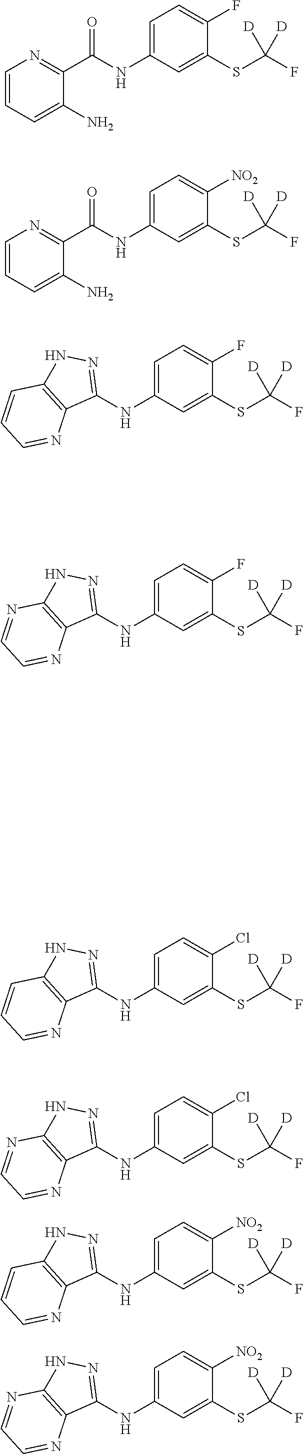

Pharmaceutically Acceptable Salts

In some embodiments, a salt of any one of the compounds of the present disclosure is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

Methods of Use

One aspect of the present application relates to compounds of formula (I) useful in imaging techniques, diagnosing and monitoring treatment of various diseases and conditions described herein. Such compounds are labeled in so far as each compound includes at least one ¹⁸F radioisotope.

Methods of Diagnosis, Imaging, and Monitoring Treatment:

mGluR4-selective PET probes of Formula (I) are noninvasive molecular-imaging tools for quantifying spatial and temporal changes in characteristic biological markers of brain disease and for assessing potential drug efficacy. In vivo imaging of mGluR4 function in normal and pathological conditions reveals new diagnostic and therapeutic strategies for CNS disorders such as PD, which are lacking cure. Hence, the compounds of Formula (I) are useful in diagnosing a neurological disease or condition, for example, by comparing the imaged brains of healthy and ill subjects. For the treating physician, this comparison may reveal important information aiding in the diagnosis. In certain embodiments, the disease is diagnosable by imaging with mGluR4 modulator of Formula (I) because mGluR4 is implicated in the pathology of the disease.

In some embodiments, the present disclosure provides a method of identifying and quantifying mGluR4 density in the brain of a subject. This may be attained, for example, by imaging the brain. A method of imaging the brain comprises (i) administering to the subject an effective amount of a compound of Formula (I), or a pharmaceutically acceptable salt thereof, or a pharmaceutical composition comprising same; (ii) waiting a time sufficient to allow the compound to accumulate in the brain to be imaged (e.g., 1 min, 5 min, 10 min, 15 min, or 30 min), and (iii) imaging the brain with an imaging technique. Since ¹⁸F within the compound of Formula (I) is positron emitting radioisotope, the suitable imaging techniques include positron emission tomography (PET) and its modification. As such, the imaging technique may be selected from positron emission tomography (PET) imaging, positron emission tomography with computer tomography (PET/CT) imaging, and positron emission tomography with magnetic resonance (PET/MRI) imaging.

In some embodiments, the present disclosure provides a method of diagnosing a neurological disorder (e.g., neurological disorder in which mGluR4 is implicated) in a subject, the method comprising (i) administering to the subject an effective amount of a compound of Formula (I), or a pharmaceutically acceptable salt thereof, or a pharmaceutical composition comprising same; (ii) waiting a time sufficient to allow the compound to accumulate in the brain to be imaged (e.g., 1 min, 5 min, 10 min, 15 min, or 30 min), and (iii) imaging the brain with an imaging technique. The method may also comprise comparing images obtained from subjects exhibiting the symptoms of the disease or condition with the images obtained from healthy subjects. In one example, loss or overabundance of mGluR4 receptors in the brain of the subject may be indicative of a neurodegenerative disease such as Parkinson's disease or a related condition.

In some embodiments, the present disclosure provides a method of supporting the clinical development of potential therapeutics, in which the receptor occupancy of potential drug candidates such as mGluR4 allosteric modulators in the brain is measured (see, e.g., ref. 36). In vivo receptor occupancy can help to answer many vital questions in the drug discovery and development process such as whether potential drugs reach their molecular targets, the relationship between therapeutic dose and receptor occupancy, the correlation between receptor occupancy and plasma drug levels, and the duration of time the drug remains at its target.

In yet other embodiments, the present disclosure provides a method of monitoring treatment of neurological disorder (e.g., neurological disorder in which mGluR4 is implicated) in a subject, the method comprising (i) administering to the subject an effective amount of a compound of Formula (I), or a pharmaceutically acceptable salt thereof, or a pharmaceutical composition comprising same, (ii) waiting a time sufficient to allow the compound of Formula (I) to accumulate in a brain of the subject (e.g., 5 min, 15 min, or 30 min); (iii) imaging the brain of the subject with an imaging technique; (iv) administering to the subject a therapeutic agent in an effective amount to treat the neurological disorder (e.g., levodopa or an experimental drug substance for treating PD); (v) after (iv), administering to the subject an effective amount of a compound of Formula (I), or a pharmaceutically acceptable salt thereof; (vi) waiting a time sufficient to allow the compound of Formula (I) to accumulate in the brain of the subject (e.g., 5 min, 15 min, or 30 min); (vii) imaging the brain of the subject with an imaging technique; and (viii) comparing the image of step (iii) and the image of step (vii). In one example, attaining overabundance of mGluR4 receptors in the brain of the subject, as determined by comparing the images, is indicative of successful treatment of the neurodegenerative disease. Suitable examples of diseases the treatment of which can be monitored according to the methods of the present disclosure include any of the diseases described herein. One particular example is PD. Other suitable examples include dyskinesia, Lewy body disease, Prion disease, motor neuron disease (MND), and Huntington's disease.

Methods of Modulating a Receptor

In some embodiments, the present disclosure provides a method of modulating (e.g., positively allosterically modulating) mGluR4 in a cell, the method comprising contacting the cell with an effective amount of a compound of the present disclosure (e.g., Formula (I) or (II)), or a pharmaceutically acceptable salt thereof, or a pharmaceutical composition comprising same. In some embodiments, the contacting occurs in vitro, in vivo, or ex vivo. In some embodiments, the cell is a neuron.

In some embodiments, the present disclosure provides a method of modulating (e.g., positively allosterically modulating) mGluR4 in a subject, the method comprising administering to the subject an effective amount of a compound of the present disclosure (e.g., Formula (I) or (II)), or a pharmaceutically acceptable salt thereof, or a pharmaceutical composition comprising same.

Methods of treating a disease or condition PD results from the progressive loss of dopaminergic neurons in the Substantia Nigra pars compacta (“SNpc”), causing dysfunction of the basal ganglia (“BG”) motor circuit. The current pharmacotherapy aims to replace missing dopamine by using the dopamine precursor levodopa (L-DOPA). This treatment provides symptomatic relief and is successful in the early PD medication period however, as the disease progresses L-DOPA becomes less effective and produces debilitating side effects such as L-dopa-induced dyskinesia (LID).

In some embodiments, the compounds and compositions of the present disclosure are useful in treating neurodegenerative disease or disorder that affects the motor system. In such embodiments, at least one mGluR (e.g., mGluR4) is implicated in the pathology of the disease or condition. One example of such disease or condition is Parkinson's disease, including associated deficits in motor system such as akinesia, bradykinesia, and dyskinesia. In some embodiments, the compounds and composition of the present disclosure are useful in treating dyskinesia, Lewy body disease, Prion disease, motor neuron disease (MND), Huntington's disease, amyotrophic lateral disorder, anxiety disorders, depression, drug addiction, pain, ischemia, ischemic brain damage, psychotic disorders related to dopaminergic/glutamatergic neuromodulation, and eating disorder. In yet other embodiments, the compounds and compositions of the present disclosure are useful in treating brain cancer. Suitable examples of brain cancer include glioblastoma and medulloblastoma. In some embodiments, the present disclosure provides a use of a compound or a composition as described herein in the manufacture of a medicament for the treatment of any one of the disease or conditions described herein.

In certain embodiments, the use of the compounds of the present disclosure prevents many common side effects of levodopa, such as nausea, vomiting, and irregular heart rhythms. Other side effects and symptoms that are reduced or eliminated by the compounds of the present disclosure include trouble falling or staying asleep, falls, and uncontrolled, involuntary movements.

Combinations

The compounds of the present disclosure can be used on combination with at least one medication or therapy useful, e.g., in treating or alleviating symptoms of PD. Suitable examples of such medications include levodopa (L-dopa), carbidopa, safinamide, dopamine agonists (e.g., ropinirole, pramipexole, rotigotine), amantadine, trihexyphenidyl, benztropine, selegiline, rasagiline, tolcapone, and entacapone, or a pharmaceutically acceptable salt thereof. The compound of the present disclosure may be administered to the patient simultaneously with the additional therapeutic agent (in the same dosage form or in different dosage forms) or consecutively (the additional therapeutic agent may be administered before or after administration of the compound of the present disclosure).

Pharmaceutical Compositions

The present application also provides pharmaceutical compositions comprising an effective amount of a compound of the present disclosure (e.g., Formula (I) or Formula (II)) disclosed herein, or a pharmaceutically acceptable salt thereof, and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

Dosages and Regimens

In the pharmaceutical compositions of the present application, a compound of the present disclosure (e.g., a compound of Formula (I) or Formula (II)) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound (e.g., Formula (I) or Formula (II)) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) or Formula (II) is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the mGluR4 with a compound of the invention includes the administration of a compound of the present invention to an individual or patient, such as a human, having mGluR4, as well as, for example, introducing a compound of the invention into a sample containing a cellular or purified preparation containing the mGluR4.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein, the term “radioisotope” refers to an atom having an atomic mass or mass number different from the atomic mass or mass number typically found in nature (i.e., naturally occurring).

As used herein, the term “isotopic enrichment factor” refers to the ratio between the isotopic abundance and the natural abundance of a specified isotope.

“D” and “d” both refer to deuterium. A compound of the present disclosure has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

“¹⁸F” refers to the radioisotope of fluorine having 9 protons and 9 neutrons. “F” refers to the stable isotope of fluorine having 9 protons and 10 neutrons (i.e., the “¹⁹F isotope”). A compound of the present disclosure has an isotopic enrichment factor for each designated ¹⁸F atom of at least 3500 (52.5% ¹⁸F incorporation at each designated ¹⁸F atom), at least 4000 (60% ¹⁸F incorporation), at least 4500 (67.5% ¹⁸F incorporation), at least 5000 (75% ¹⁸F), at least 5500 (82.5% ¹⁸F incorporation), at least 6000 (90% ¹⁸F incorporation), at least 6333.3 (95% ¹⁸F incorporation), at least 6466.7 (97% ¹⁸F incorporation), at least 6600 (99% ¹⁸F incorporation), or at least 6633.3 (99.5% ¹⁸F incorporation).

### Examples

Materials and Methods. All reagents and starting materials were obtained from the commercial sources including Sigma-Aldrich (St. Louis, Mo.), Thermo Fisher Scientific, Oakwood Products, Inc., Matrix Scientific and used as received. The reactions were monitored by TLC using a UV lamp monitored at 254 nm. If necessary, the reactions were also checked by LC-MS using the Agilent 1200 series HPLC system coupled with a multiwavelength UV detector and a model 6310 ion trap mass spectrometer (Santa Clara, Calif.) equipped with a Luna C₁₈ column (Phenomenex, 100×2 mm, 5 μm, 100 Å). The RP-HPLC was carried out by using a 7-min gradient method (LC-Method A1): eluent A: 0.1% formic acid/H₂O; eluent B: 0.1% formic acid/CH₃CN; gradient: 5% B to 95% B from 0 to 3 min, 95% B from 3 to 4.5 min, 95% to 5% B from 4.5 to 5 min, 5% B from 5 to 7 min; flow rate at 0.7 mL/min. The silica gel used in flash column chromatography was from Aldrich (Cat. 60737, pore size 60 Å, 230-400 mesh). The products were identified by LC-MS as well as ¹H NMR, ¹³C NMR and ¹⁹F NMR using a Varian 500 MHz spectrometer. All NMR samples were dissolved in chloroform-d (CDCl₃) containing tetramethylsilane as a reference standard. Chemical shifts were expressed as ppm and calculated downfield or upfield from the NMR signal of reference standard. J was expressed as Hz, and its splitting patterns were reported as s, d, t, q, or m. HRMS was acquired using a DART-SVP ion source (IonSense, Saugus, Mass.) attached to a JEOL AccuTOF 4G LC-plus mass spectrometer (JEOL USA, Peabody, Mass.) in positive-ion mode from Prof. Peter Caravan's Laboratory. Unless otherwise specified, the purities of all new compounds were over 95% determined by HPLC.

Animal procedures. The animal studies were approved and done under strict supervision of Subcommittee on Research Animals of the Massachusetts General Hospital and Harvard Medical School and performed in accordance with the Guide of NIH for the Care and Use of Laboratory Animals.

Determination of Log D. An aliquot (10 μL, 74 kBq) of [¹⁸F]15 was added to a test tube containing 2.5 mL of octanol and 2.5 mL of phosphate buffer solution (pH 7.4). The test tube was mixed by vortex for 2 min and then centrifuged for 2 min to fully separate the aqueous and organic phase. The samples taken from the octanol layer (0.1 mL) and the aqueous layer (1.0 mL) were saved for radioactivity measurement. An additional aliquot of the octanol layer (2.0 mL) was carefully transferred to a new test tube containing 0.5 mL of octanol and 2.5 mL of phosphate buffer solution (pH 7.4). The previous procedure (vortex mixing, centrifugation, sampling, and transfer to the next test tube) was repeated until six sets of aliquot samples had been prepared. The radioactivity of each sample was measured using PerkinElmer Wizard2 2480 gamma-counter. The log D of each set of samples was calculated as the following:

Log D_{7.4}=Log(radioactivity of octanol layer×10/radioactivity of PBS layer)

Plasma Protein Binding Assay. An aliquot of radiolabeled compound [¹⁸F]15 in saline (10 μL, 74 kBq) was added to a sample of human plasma (0.5 mL). The mixture was gently mixed by repeated inversion and incubated for 10 min at room temperature. Following incubation, a small sample (10 μL) was removed to determine the total radioactivity in the plasma sample (A_{T}; A_{T}=A_{bound}+A_{unbound}). The upper part of the Centrifree tube was discarded, and an aliquot (10 μL) from the bottom part of the tube was removed to determine the amount of unbound radioactivity (A_{unbound}) that passed through the membrane (molecular weight cutoff 30 kD). The radioactivity of each sample was measured using PerkinElmer Wizard2 2480 gamma-counter. Plasma protein binding was derived by the following equation: % unbound=A_{unbound}×100/A_{T}.

Metabolic stability. The stability of test compounds was measured using rat liver microsomes and a literature method (Drug Metab Dispos. 1999; 27(11): 1350-9). The incubation mixtures were consisted of rat liver microsomes (0.5 mg/ml), test compounds (5.0 μM in DMSO stock solution), and NADPH (1.3 mM) in 0.5 ml of potassium phosphate buffer (25 mM, pH 7.5). The microsomes were incubated in a vial for 3 min at 37° C., and then the test compound and NADPH were added into the vial to start the reaction. The mixture was shaken in a water bath at 37° C. At different time points, aliquots (50 μL) were removed and added 100 μI of cold acetonitrile and the internal standard (10.0 μM) to quench the reaction. The precipitation was removed by centrifugation (10,000 g, 10 min, 4° C.), and the supernatant was analyzed by RP-HPLC. The percentage of remaining intact test-compound was calculated by (peak area at 60 min)/(peak area at 0 min)×100%. Each procedure was repeated three times.

Ex vivo Studies of Biodistribution. For biodistribution studies the normal male Sprague Dawley rats were anesthetized with isoflurane/nitrous oxide (1-1.5% isoflurane) to install catheter into the tail vain for administration of radioactivity ([¹⁸F]15, 22±3 MBq, iv). Total of 15 rats in deep anesthesia (isoflurane 4% and cervical dislocation) were sacrificed in a group of three at five different time points (5, 10, 20, 30, and 60 min) after injection of [¹⁸F]15. Major organs including lung, heart, liver, spleen, kidney, muscle, midbrain, cortex, cerebellum and blood were harvested to determine radioactivity. The tissue samples were weighted, and the radioactivity was measured with the standard samples of [¹⁸F]15 using Wizard2 2480, Perkin Elmer, Calif. Radioactivity of the tissue samples was determined as percent of the injected activity per gram of the tissue.

PET Imaging of [¹⁸F]15 in Rats. Altogether 24 normal male Sprague Dawley rats were used for the imaging studies comprising 15 baseline studies and 15 blocking studies. Rats were anaesthetized with isoflurane/nitrous oxide (1.0-1.5% isoflurane, with oxygen flow of 1-1.5 L/min), and catheter was installed into tail vein for the administration of [¹⁸F]15. Dynamic volumetric PET data was acquired with a PET-CT scanner for 60 min (Triumph-II, Tri-Foil Imaging, Northridge, Calif.). The vital signals such as heart rate and respiration rate were monitored during scanning period. PET data acquisition was started immediately after administration of radioactivity with the dose range of 18-34 MBq depending on the size of the rat followed by CT imaging to obtain anatomical information and data for attenuation correction of PET data. PET data was processed by using maximum-likelihood expectation-maximization (MLEM) algorithm with 30 iterations to dynamic volumetric images, and corrected for uniformity, scatter, and attenuation. The CT data was processed by the modified Feldkamp algorithm using matrix volumes of 512×512×512 and pixel size of 170 m. Co-registration of CT and PET images and analysis of PET images were carried out using PMOD3.2 software (PMOD Technology, Zurich, Switzerland).

For blocking studies, the same scanning protocols were used as for the baseline studies. For mGluR4 blocking experiments, 1, 2 or 3 mg/kg of 13 or 15 dissolved in a saline solution with 10% DMSO, 5% Tween-20 and 85% PBS was injected 1 min before iv administration of [¹⁸F]15.

### Example 1—Synthesis of Fluoromethyl-d2 4-Methylbenzenesulfonate (17)

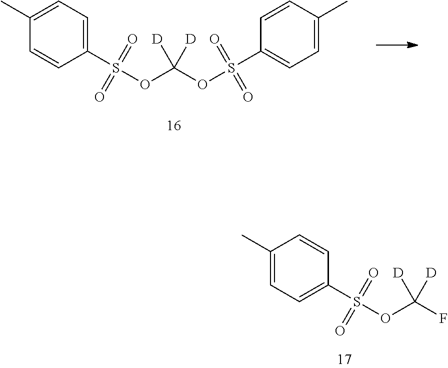

To the solution of methylene-d2 bis(tosylate) (16, 9.01 g, 25.2 mmol) in tert-amyl alcohol (150 mL) was added CsF (3.83 g, 25.2 mmol). The mixture was stirred at 80° C. for 2.0 h. A large amount of white solid precipitated out. After filtration, the filtrate was condensed under vacuum. The resulting residue was chromatographed on silica gel by eluting with ethyl acetate and hexane (1:30 to 1:15) to afford 17 as a colorless oil (2.49 g, 48%) and 3.02 g of starting material 16 was recovered. ¹H NMR (500 MHz, CDCl₃) δ 7.83 (d, J=8.0 Hz, 2H), 7.36 (d, J=8.0 Hz, 2H), 2.46 (s, 3H). ¹³C NMR (126 MHz, CDCl₃) δ 145.6, 133.8, 129.9 (2C), 127.9 (2C), 97.6 (dp, J=26.5 Hz, J=229.3 Hz), 21.7. ¹⁹F NMR (470 MHz, CDCl₃) δ −154.5 (p, J=9.4 Hz). LC-MS (method A1): t_{R}=3.67 min, (ESI) m/z calcd. for C₈H₂₂D₂FNO₃S 224.0; found 224.0 [M+NH₄]⁺. Compound 17 was previously made from 16 in 5% yield by using TBAF, 80° C., in MeCN and t-BuOH for 2 h.

### Example 2—N-(4-Chloro-3-((fluoromethyl-d2)thio)phenyl)picolinamide (15)

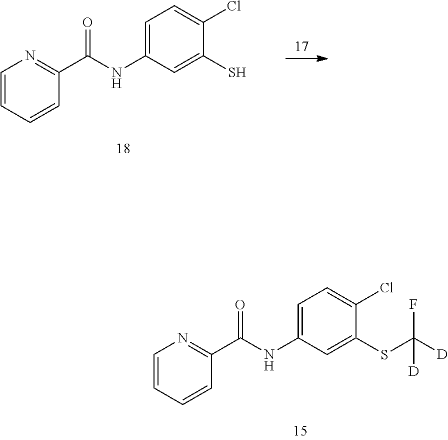 [SMILES: O=C(Nc1ccc(Cl)c(S)c1)c1ccccn1 ; [2H]C([2H])(F)Sc1cc(NC(=O)c2ccccn2)ccc1Cl]

To the solution of N-(4-chloro-3-mercaptophenyl)picolinamide hydrogen chloride salt (18, 150.0 mg, 0.5 mmol) and 17 (154.5 mg, 0.75 mmol) in acetonitrile (7.0 mL) were added K₂CO₃ (690 mg, 5.0 mmol) and KI (83.0 mg, 0.5 mmol). The resulting mixture was heated to reflux for 2 h. The solvent was removed under vacuum and the residue was dissolved in DCM (20 mL) and water (20 mL). The water phase was further washed with DCM twice (20 mL). The organic layer was combined, dried over anhydrous Na₂SO₄ and concentrated under vacuum. The residue was chromatographed on silica gel by eluting with EtOAc and hexane (1:3) to afford the title product as a white powder (137.2 mg, 92%). ¹H NMR (500 MHz, CDCl₃) δ 10.08 (s, 1H), 8.62 (ddd, J=4.7, 1.6, 0.9 Hz, 1H), 8.29 (dt, J=7.8, 1.0 Hz, 1H), 7.92 (td, J=7.7, 1.7 Hz, 1H), 7.86 (d, J=1.7 Hz, 1H), 7.51 (ddd, J=7.6, 4.7, 1.2 Hz, 1H), 7.44 (dd, J=8.7, 2.3 Hz, 1H), 7.39 (d, J=8.6 Hz, 1H). ¹⁹F NMR (470 MHz, CDCl₃) δ −150.9 (dt, J=16.2, 8.2 Hz). ¹³C NMR (126 MHz, CDCl₃) δ 162.1, 149.3, 148.0, 137.8, 137.3, 134.3, 130.2, 128.62 (d, J=2.4 Hz), 126.7, 122.5, 120.6, 119.5, 85.8 (dp, J=24.0, 217.2 Hz). LC-MS (method A1): t_{R}=4.05 min, (ESI) m/z calcd. for C₁₃H₉D₂ClFN₂OS 299.0; found 298.9 [M+H]⁺. HRMS (m/z) calcd. for C₁₃H₉D₂ClFN₂OS 299.0390; found 299.0388 [M+H]⁺.

### Example 3—N-(4-chloro-3-mercaptophenyl)picolinamide (18)

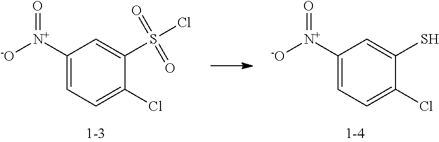

2-Chloro-5-nitrobenzenesulfonyl chloride (1-3, 2.55 g, 10.0 mmol) was dissolved in 50 ml of toluene, which was heated to reflux. PPh₃ (7.86 g, 30.0 mmol) was added slowly over 10 min to the mixture until 2-chloro-5-nitrobenzenesulfonyl chloride was consumed. The crude mixture was cooled to room temperature then concentrated under vacuum. The residue was chromatographed on silica gel eluting with ethyl acetate and hexane (1:5) to afford product 1-4 as yellow crystals (1.72 g, 91%). ¹H NMR (500 MHz, CDCl₃) δ 8.46 (d, J=2.6 Hz, 1H), 8.07 (dd, J=8.7, 2.6 Hz, 1H), 7.60 (d, J=8.7 Hz, 1H). ¹³C NMR (126 MHz, CDCl₃) δ 147.4, 138.7, 136.5, 130.9, 123.1, 122.3. HPLC trace (Method A1): t_{R}=3.98 min, mass of 1-4 was not detectable.

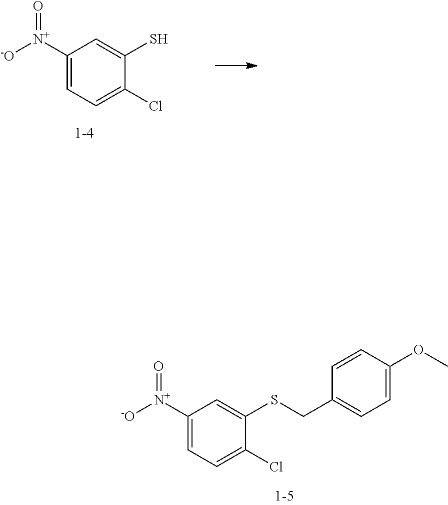

To the solution of 2-chloro-5-nitrobenzenethiol (1-4, 1.33 g, 7.0 mmol) in acetonitrile (20 ml) was added K₂CO₃ (2.76 g, 20.0 mmol), KI (1.20 g, 7.2 mmol) and 4-methoxybenzyl chloride (1.50 g, 9.6 mmol). The mixture was refluxed for 2 h. The crude mixture was cooled to room temperature then concentrated under vacuum. The residue was dissolved in DCM (20 ml) and water (20 ml). The water phase was further washed with DCM twice (20 mL). The organic extracts were dried over anhydrous Na₂SO₄ and concentrated under vacuum. The crude product was chromatographed on silica gel eluting with ethyl acetate and hexane (1:7) to afford product 1-5 as pale-yellow needle crystals (2.11 g, 97%). ¹H NMR (500 MHz, CDCl₃) δ 8.12 (d, J=2.6 Hz, 1H), 7.92 (dd, J=8.7, 2.6 Hz, 1H), 7.50 (d, J=8.7 Hz, 1H), 7.35 (d, J=8.7 Hz, 2H), 6.88 (d, J=8.7 Hz, 2H), 4.22 (s, 2H), 3.80 (s, 3H). ¹³C NMR (126 MHz, CDCl₃) δ 159.3, 146.7, 139.5, 138.8, 130.2, 130.0, 126.5, 121.8, 120.6, 114.3, 55.3, 36.6. LC-MS (method A1): t_{R}=4.50 min, (ESI) m/z calcd for C₁₄H₁₂CINO₃S 309.0, found 327.0 [M+NH₄]⁺; 331.9 [M+Na]⁺.

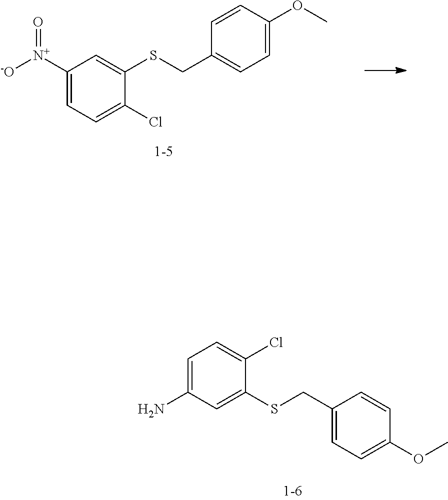

To the solution of (2-chloro-5-nitrophenyl)(4-methoxybenzyl)sulfane (1-5, 1.55 g 5.0 mmol) and NiCl₂.6H₂O (118.0 mg, 0.5 mmol in 2.0 mL of MeOH) in 20 mL of THF was added NaBH₄ (567.0 mg, 15.0 mmol). The reaction was slowly heated to reflux until the starting material was consumed (10 min). The crude mixture was cooled to room temperature then concentrated under vacuum. 20 mL of distilled water and 30 mL of ethyl acetate were added to the residue and the water phase was further washed with ethyl acetate (2×30 mL). The organic extracts were dried over anhydrous Na₂SO₄ and concentrated under vacuum. The residue was chromatographed on silica gel eluting with ethyl acetate and hexane (1:2) to afford product 1-6 as a white solid (1.29 g, 92%). ¹H NMR (500 MHz, CDCl₃) δ 7.27 (d, J=6.95 Hz, 2H), 7.12 (d, J=8.5 Hz, 1H), 6.84 (d, J=8.7 Hz, 2H), 6.57 (dd, J=8.5, 2.7 Hz, 1H), 6.44 (dd, J=8.5, 2.7 Hz, 1H), 4.07 (s, 2H), 3.79 (s, 3H). ¹³C NMR (126 MHz, CDCl₃) 158.9, 145.3, 136.3, 130.1, 130.0, 129.9, 128.3, 122.7, 115.6, 114.0, 113.8, 55.3, 37.0. LC-MS (method A1): t_{R}=4.00 min, (ESI) m/z calcd for C₁₄H₁₄CINOS 279.1, found 280.0 [M+H]⁺.

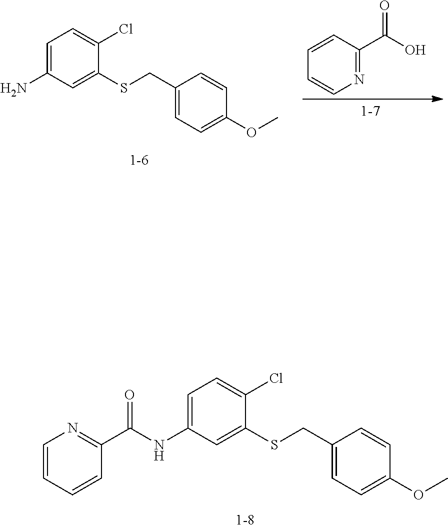 [SMILES: COc1ccc(CSc2cc(N)ccc2Cl)cc1 ; O=C(O)c1ccccn1 ; COc1ccc(CSc2cc(NC(=O)c3ccccn3)ccc2Cl)cc1]

To the solution of picolinic acid (1-7, 152.2 mg, 1.24 mmol) and 4-chloro-3-((4-methoxybenzyl)thio)aniline (1-6, 279.0 mg, 1.0 mmol) in DCM (12 ml) were added N,N′-Diisopropylcarbodiimide (252.7 mg, 2.0 mmol) and 4-dimethylaminopyridine (244.5, 2.0 mmol). The resulting mixture was stirred at room temperature overnight. The reaction mixture was then filtered, and the filtrate was dried under vacuum. The residue was chromatographed on silica gel eluting with ethylacetate and hexane (1:2) to afford product 1-8 as white needle crystals (345.2 mg, 90%). ¹H NMR (500 MHz, CDCl₃) δ 10.03 (s, 1H), 8.63 (ddd, J=4.8, 1.7, 0.9 Hz, 1H), 8.30 (dd, J=7.8, 1.0 Hz, 1H), 7.97 (d, J=2.3 Hz, 1H), 7.94 (tdd, J=7.8, 1.6, 1.0 Hz, 1H), 7.56-7.48 (m, 1H), 7.44 (ddd, J=8.6, 2.4, 0.7 Hz, 1H), 7.36 (dd, J=6.7, 1.8 Hz, 2H), 7.33 (s, 1H), 6.86 (d, J=8.1 Hz, 2H), 4.20 (s, 2H), 3.79 (d, J=0.9 Hz, 3H). ¹³C NMR (126 MHz, CDCl₃) δ 162.0, 159.0, 149.4, 148.0, 137.8, 137.3, 136.9, 130.3, 129.8, 127.8, 127.8, 126.7, 122.4, 119.1, 117.6, 114.0, 55.3, 36.9. LC-MS (method A1): t_{R}=4.53 min, (ESI) m/z calcd. for C₂₀H₁₁CIN₂O₂S 384.1, found 385.0 [M+H]+.

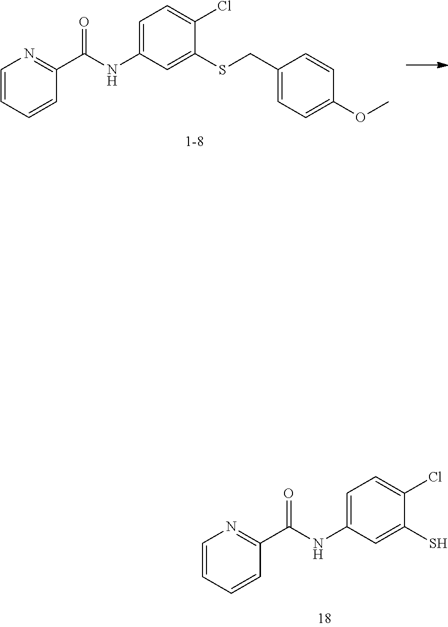 [SMILES: COc1ccc(CSc2cc(NC(=O)c3ccccn3)ccc2Cl)cc1 ; O=C(Nc1ccc(Cl)c(S)c1)c1ccccn1]

Compound N-(4-chloro-3-((4-methoxybenzyl)thio)phenyl)picolinamide (1-8, 269.0 mg, 0.7 mmol) was dissolved in TFA (5.0 mL). The mixture was refluxed for 2 h. The solvent was removed under vacuum and the residue was recrystallized from 5 mL of MeOH containing hydrogen chloride (37%, 0.12 mL) to give product 1-9 as a white solid (203.7 mg, 97%). ¹H NMR (500 MHz, DMSO-d6) δ 10.74 (s, 1H), 8.74 (s, 1H), 8.21 (d, J=1.9 Hz, 1H), 8.15 (d, J=7.3 Hz, 1H), 8.08 (t, J=7.5 Hz, 1H), 7.69 (s, 1H), 7.61 (dd, J=8.7, 2.0 Hz, 1H), 7.41 (d, J=8.7 Hz, 1H), 5.80 (s, 1H). ¹H NMR (500 MHz, CD₃OD) δ 8.41 (br, 1H), 7.90 (br, 1H), 7.73 (br, 1H), 7.69 (s, 1H), 7.32 (br, 1H), 7.26 (d, J=7.3 Hz, 1H), 7.06 (d, J=8.6 Hz, 1H). ¹³C NMR (126 MHz, DMSO-d6) δ 163.1, 150.0, 148.9, 138.7, 138.0, 133.3, 129.9, 127.6, 125.6, 123.0, 121.6, 119.3. LC-MS (method A1): t_{R}=4.09 min, (ESI) m/z calcd. for C₁₂H₉CIN₂OS 264.0, found 265.0 [M+H]+.

### Summary of Examples 1-3

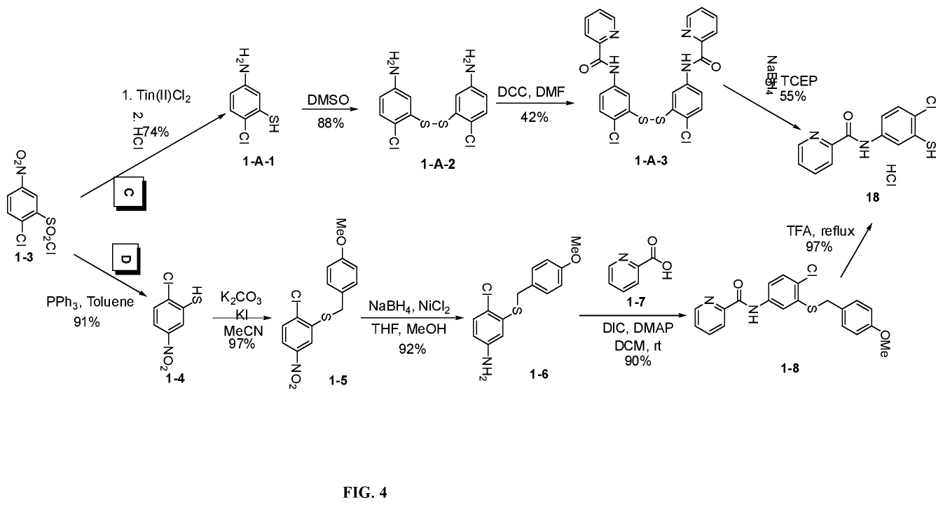

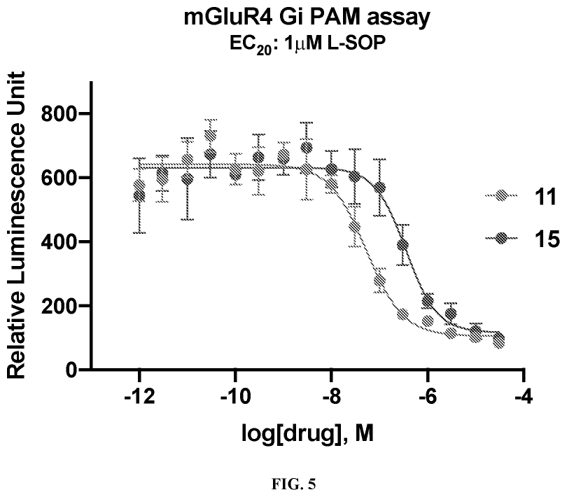

The key intermediate compound 18 was previously synthesized from compound 1-3 via 4-step reactions (process C) in only 15% total yield (see ref 34). As the Examples 1-3 show, the synthesis of compound 18 was efficiently achieved through 5-step reactions (process D) in 71% total yield. See FIG. 4. The current process can be summarized as follows. The reaction of methylene-d2 bis(tosylate) 16 with cesium fluoride was carried out in t-amyl alcohol at 80° C. to give fluoromethyl-d₂ 4-methylbenzenesulfonate 17 in 48% yield.

The choice of solvent used in this reaction had influence on the reaction yield. If the reaction was carried out in acetonitrile, the yield was 5%. The synthesis of compound 15 was achieved by treating 17 with the thiophenol precursor 18 in 92% yield

To evaluate compound 15, the pharmacochemical properties including affinity to mGluR4, mGluR4 PAM activity, selectivity to other mGluRs, lipophilicity, plasma protein binding, metabolic and solution stabilities were determined. In addition, compound 15 was compared to the previously reported mGluR4 PET ligands; compounds 4, 13 and others.

### Example 4—Radioligand Replacement Assay

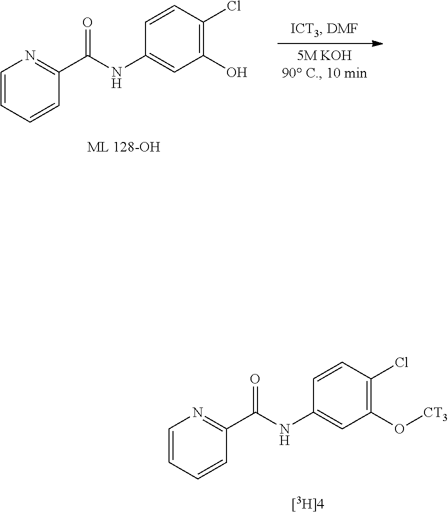

The affinity determined by the radioligand replacement assay. Synthesis of [³H]4. [³H]Iodomethane (25 mCi, 1 Ci/mL in DMF, American Radiolabeled Chemicals Inc.) was added to a solution of ML128-OH (1.5 mg) and 5M potassium hydroxide solution (3.0 μL) in 0.3 mL of DMF. The reaction mixture was heated at 90° C. for 10 min and diluted with 1.0 mL of HPLC solvents. Then, the aliquot was injected into HPLC equipped with Gemini-NX C18 semi-preparative column (250 mm×10 mm, 5μ, Phenomenex Inc.), flow scintillation detector, and internal UV detector eluting with a solution of 55% acetonitrile and 45% 0.1 M ammonium formate at a flow rate of 4 mlLmin. The fractions containing the radiolabeled compound [³H]4 were collected between 11-13 min. The radioactive product was diluted with 30 mL of water and passed through C18 Sap-Pak Plus followed by additional wash with 5 mL of sterile water. Finally, total 6.34 mCi of [³H]4 was eluted from cartridge with ethanol (25% yield). The radioactivity was measured, and the final concentration was set as 10 μM in EtOH and stored in vial at −20° C. during the experiments. The chemical identity of [³H]4 was confirmed by injection with the cold reference compound into radio-HPLC.

Compounds 4, 8, 13 and 15 were characterized with the competitive binding assay using mGluR4 transfected CHO cells by increasing the concentration of test compound from 0.01 nM to 10 μM in presence of 2 nM of [³H]4, in which the binding affinities to mGluR4 were described as IC₅₀ values (Table 1)

TABLE 1
Affinity to mGluR4.

| Compound | 4 | 8 | 13 | 15 |
|---|---|---|---|---|
| IC₅₀ (nM) | 5.1 | 3.2 | 3.4 | 3.4 |

As displayed in Table 1, compound 15 has a similar IC₅₀ value (3.4 nM) as that of 8 (3.2 nM) and 13 (3.4 nM) and has enhanced affinity compared to 4 (5.1 nM). The results also confirmed that compound 15 binds to the same allosteric site of mGluR4 as that of the other three compounds.

In Vitro mGluR4 Binding Assay. CHO cells expressing mGluR4 were used for all binding assay protocols. For competitive binding, 2 nM of [³H]4 was used together with increased concentrations of test compounds ranging from 0.01 nM to 10 μM. Each test tube contained 50 000 freshly harvested CHO cells expressing mGluR4 cultured in HAMs cell culture medium with 100 nM of glutamate, penicillin-streptomycin (100 units), and 1 mM G418. [3H]4 (2 nM) was added to the cell extract with and without test compounds on ice and then incubated for 30 min at room temperature. Samples were centrifuged at 1200 rpm for 10 min at 4° C. and washed three times with a cold cell culture medium as a washing buffer. Samples were lysed by adding 100 μL of 0.5% NaOH and heated using a heating block (56° C., 30 min). Samples were cooled with an ice bath and transferred to Solvent-Saver scintillation vials (VWR International LLC.). To obtain binding parameters, the scintillation liquid (PerkinElmer, Optima Gold) was added prior to counting with a scintillation counter (Packard TriCarb Model, 1 min/vial). Nonspecific binding was determined using 10 μM of nonradioactive test compound, and specific binding was determined by extracting the nonspecific binding from total binding. All measurements were done in triplicate and analyzed with GraphPad Prism software (GraphPad Software Inc.).

### Example 5—mGluR4 Functional Activity Assay

Cell Culture. HEK-293 cells were maintained with complete Dulbecco's modified Eagle's medium (DMEM), which was composed of 10% fetal bovine serum (FBS), 2 mM L-glutamine, 100 units/mL penicillin G, 100 μg/mL streptomycin at 37° C. in the presence of 5% CO₂. HEK-293 stable cell lines with tetracycline inducible expression of mGluR1, mGluR2, mGluR4, mGluR6 or mGluR8 were maintained with complete DMEM with Hygromycin B (100 μg/mL), Blasticidin (15 μg/mL) at 37° C. in the presence of 5% CO₂.

Ca²⁺ mobilization assay. The Gq coupled receptors (mGluR1 and mGluR5) were tested using Ca²⁺ mobilization assay. mGluR1 stable cell lines were plated into poly-L-lysine (PLL) coated 384-well black clear bottom cell culture plates with complete Basal Medium Eagle (BME) buffer, which was composed of 10% dialyzed FBS, penicillin G (100 units/mL), streptomycin (100 μg/mL) with Tetracycline (1 μg/mL) at density of 20,000 cells in 40 μl per well for overnight. On the other hand, HEK-293 Cells transiently transfected using the calcium phosphate method with cDNA encoding mGluR5 for 40 h were plated into the plate with complete BME at density of 20,000 cells in 40 μL per well for 8 h. mGluR1 stable cells or cells transiently expressing mGluR5 were incubated with 20 μL of the calcium dye (FLIPR Calcium 4 Assay Kit; Molecular Devices) diluted in the assay buffer (1×HBSS, 2.5 mM probenecid, and 20 mM HEPES, pH 7.4) for 45 min at 37° C. and 15 min at room temperature. To measure agonist activity of receptors, the drug plates were prepared with different concentrations of test or reference compound at 3 times the desired final concentration. When measuring antagonist activity, another drug plate which contained EC₈₀ concentration of the reference drug was prepared. Once loaded in FLIPR (Molecular Devices), basal fluorescence was measured for 10 s, then 10 μL of test or reference compounds were added, followed by continued fluorescence measurement for an additional 120 s. Raw data were plotted as a function of molar concentration of the compound with Prism 5.0 (GraphPad Software).

cAMP assay. The Gi/o coupled receptors (mGluR2, mGluR3, mGluR4, mGluR6 and mGluR8) were tested using cAMP assay. Promega's split luciferase based GloSensor cAMP biosensor technology was used in determining Gi-GPCR mediated cAMP production in live cells. On the cells stably expressing mGluR2, mGluR3, mGluR4, mGluR6 or mGluR8, GloSensor cAMP DNA construct was transfected overnight. Cells were seeded into PLL coated 384-well white clear bottom cell culture plates with complete BME Buffer with Tetracycline (1 μg/mL) at a density of 20,000 cells for another 24 h. The cell medium was removed and then 20 μL of buffer was loaded. To measure the agonist activity, 10 μL of 3× test compound solution was added for 15 min before addition of 10 μL of luciferin/isoproterenol mixture at a final concentration of 4 mM and 200 nM, respectively, followed by counting of the plate. To measure the PAM or antagonist activity, cells were pre-incubated with test compound for 15 min before addition of EC₂O or EC₈₀ concentration of a reference agonist for another 15 min. Then 10 μL of luciferin/isoproterenol mixture at a final concentration of 4 mM and 200 nM, respectively, was added for 15 min followed by counting of the plate. In these experiments, isoproterenol was used to activate endogenous β₂ adrenergic receptors expressed in HEK293 T cells to activate the endogenous Gs protein. Luminescence was counted in a Trilux luminescence counter. Data were analyzed with Prizm 5.0 (GraphPad software).

Secondary assays—Dose-response assays. Compounds were tested for their potency in dose-response experiments. Eight-point dose response curves were performed in duplicate twice on two separate lots of cells (sometimes a third curve might be needed if in the first experiment the range of concentrations used was outside of the active range). For antagonists, these curves were performed in the presence of the EC₈₀ concentration of the agonist. For each compound, the results from four replicates were averaged and then either EC₅₀ or IC₅₀ values were calculated by non-linear regression using the 4-parameter logistic equation. Results were reported as EC₅₀ or IC₅₀ values for each tested compound (and receptor) and include the EC₅₀ or IC₅₀ values of a known agonist or antagonist for comparison purposes.

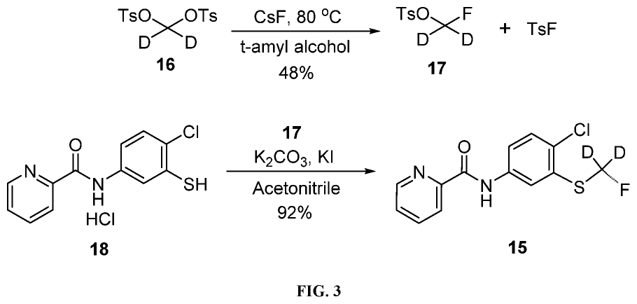

In sum, the mGluR4 PAM activity was determined using Promega's split luciferase based GloSensor cAMP biosensor assay (refs. 41-42). mGluR4 is coupling to Gαi protein to inhibit adenylate cyclase, which converts ATP to cAMP, which plays an important role in many signal transduction pathways. Consequently, changes in the intracellular cAMP levels correlate with the degree of GPCR activation and measurement of intracellular cAMP levels is a well-established approach to GPCRs in drug discovery. The mGluR4 PAM activity of 15 was evaluated in presence of EC₂₀ concentration of agonist (1 μM L-SOP) by measuring changes in intracellular cAMP concentration. Compound 11 (TC-N 22 Å), one of the most potent mGluR4 PAMs, was used as the reference compound for the assay. As shown in FIG. 3, the EC₅₀ values of 11 and 15 were 55 nM and 324 nM, respectively, showing that 15 is a potent mGluR4 PAM.

mGluR Subtype Selectivity

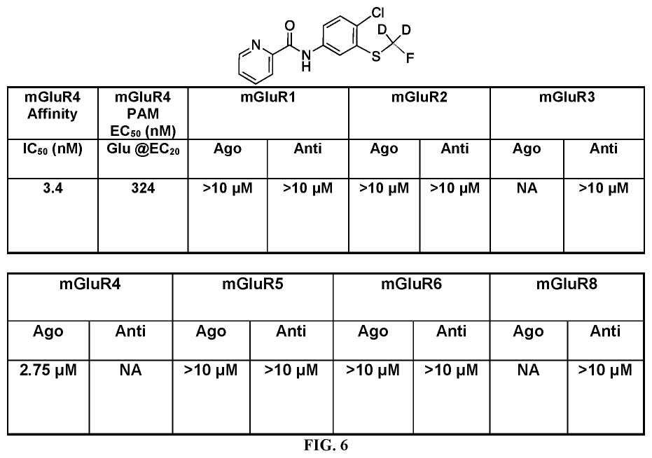

The selectivity of 15 was also analyzed among the various mGluR subtypes, in which the Gq coupled receptors (mGluR1 and mGluR5) were tested using Ca²⁺ mobilization assay and the Gi/o coupled receptors (mGluR2, mGluR3, mGluR4, mGluR6 and mGluR8) using the cAMP assay. Results demonstrated that 15 has selectivity against other mGlu receptors (FIG. 6) and that 15 has mGluR4 agonist activity (EC₅₀=2.75 μM), hence, 15 is an mGluR4 ago-PAM.

### Example 6—Pharmacochemical Properties

Lipophilicity has a major effect on BBB penetration, ADMET properties and pharmacological activity. The values of lipophilicity (c Log P) and tPSA for 15 were calculated by using ChemDraw as 2.95 and 41.46, respectively. The Log D_{7.4} of 15 was 3.82 as measured with a scaled-down shake flask method using [¹⁸F]15 (Table 2) and is in the range normally considered favorable for a PET ligand. (Ref. 43).

TABLE 2
Pharmacochemical properties.

| Compound | MW | tPSA | HBD | cLogP | Log D_{7.4} | PPB |
|---|---|---|---|---|---|---|
| 15 | 298.76 | 41.46 | 1 | 2.95 | 3.82 | 93.1% |

As the PET tracer is delivered into the bloodstream, it can bind to albumin, α1-acid glycoprotein and lipoproteins. Plasma protein binding reduces the free drug in the bloodstream and inhibits BBB penetration to reach the brain target. (Ref 44) The PPB value was obtained with an ultrafiltration assay by using [¹⁸F]15. As Table 2 shows, the plasma protein binding of 15 was 93.1%, which gives a suitable free tracer concentration available to cross the BBB. These pharmacological properties along with other molecular properties such as MW, tPSA and HBO are in the range normally considered favorable for a PET ligand.

### Example 7—Plasma Stability

Briefly, each tube is added 882 μL of 1:1 diluted rat plasma with phosphate buffer (pH 7.4). 18 μL (100 μM) of DMSO stock solution of the test compound (4, 13 and 15) or positive control compound (Diltiazem) was added to make a final compound concentration 2 μM in the plasma solution and final DMSO content 2%. The mixture was incubated at 37° C. After the incubation at 0, 15, 30, 60, and 120 min time points, aliquots of 50 μL samples were quenched with 100 μL of ice-cold acetonitrile with 25 μM of the internal reference (for HPLC analysis), respectively. The quenched samples were centrifuged at 10,000×g, and the supernatant was analyzed by HPLC. The percentage of remaining intact test compound was calculated by (peak area at the specific time point)/(peak area at 0 min)×100. Every sample was assayed three times.

TABLE 3
Plasma stability.

| Time | | | | |
|---|---|---|---|---|
| (min) | mG4P005 | mG4P0012 | 15 | Diltiazem |
| 0 | 100.00 ± 4.65 | 100.00 ± 0.21 | 100.00 ± 1.88 | 100.00 ± 8.98 |
| 15 | 94.45 ± 1.65 | 95.34 ± 12.08 | 93.46 ± 1.96 | 87.33 ± 9.53 |
| 30 | 97.56 ± 11.8 | 95.27 ± 7.76 | 104.21 ± 2.84 | 75.23 ± 4.03 |
| 60 | 100.35 ± 8.04 | 93.62 ± 8.76 | 94.54 ± 5.18 | 58.91 ± 13.39 |
| 120 | 98.21 ± 6.32 | 91.52 ± 2.49 | 84.52 ± 10.16 | 48.97 ± 10.63 |

^{a}The remaining of the intact test compounds.

### Example 8—Microsomal Stability

First, Sprague-Dawley rat liver microsomes (0.5 mg/mL, Sigma-Aldrich, No. M9066) was incubated with PBS (50 mM, pH=7.4) in a reaction tube at 37° C. for 3 min. To the reaction tube were added the test compound (2 μM) and then cofactor, NADPH (1 mM), to start the reaction. The mixture was shaken in a water bath at 37° C. At different time points (0, 5, 10, 15, 30 min), aliquots (50 μL) were removed and added to 100 μl of cold acetonitrile with internal standard (25 μM) to quench the reaction. The precipitated material was by centrifugation (10,000×g, 10 min, 4° C.), and the supernatant was analyzed by RP-HPLC (Column: Phenomenex Luna© 5 μm, Cis, 100 Å, 250×4.6 mm). The RP-HPLC was carried out by using a 15-min gradient method: eluent A: 0.1% formic acid/H₂O; eluent B: 0.1% formic acid/CH₃CN; gradient: 5% B to 95% B from 0 to 1 min; 5% B to 95% B from 1 to 9 min, 95% B from 9 to 12 min, 95% to 5% B from 12 to 13 min, 5% B from 13 to 15 min; flow rate at 0.7 mL/min.

The in peak area ratio (test compound peak area at a time point/peak area at 0 min time point) was plotted against time and the gradient of the line was determined. Subsequently, the half-life (t_{1/2}) was calculated as 0.693/k, where the elimination rate constant k equals to −gradient. V (μL/mg) was obtained as a ratio of volume of incubation (L)/protein in the incubation (mg). Finally, the intrinsic clearance (CLint) (μL/min/mg protein) was calculated as V*0.693/t_{1/2}.

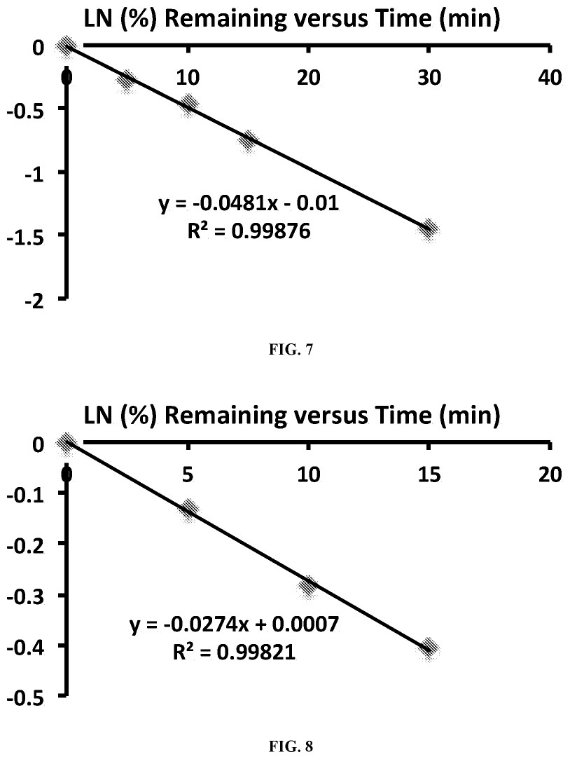

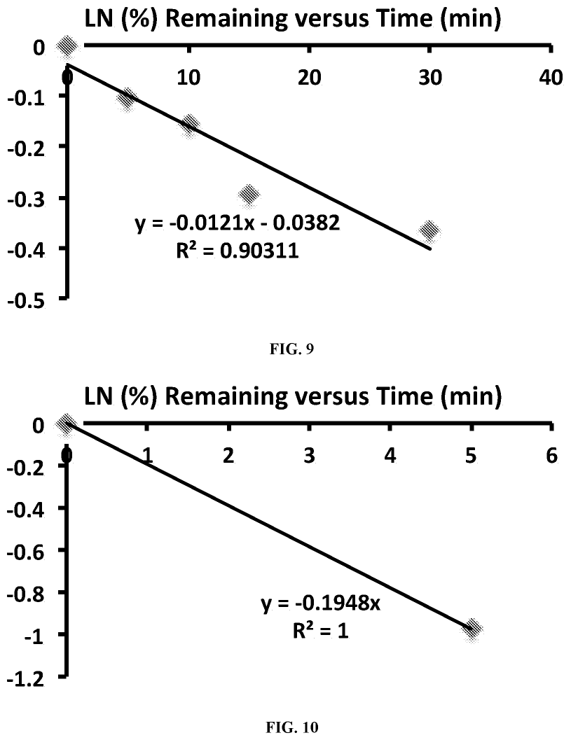

FIG. 7 shows microsomal stability of compound 4; t₁/2=0.693/(−gradient)=14.41 min; V (uL/mg)=V*0.693/t½=V*(−gradient)=96.2 μL/min/mg protein. FIG. 8 shows microsomal stability of compound 13; t₁/2=0.693/(−gradient)=25.30 min; V (μL/mg)=V*0.693/t½=V*(−gradient)=54.8 μL/min/mg protein. FIG. 9 shows microsomal stability of compound 15; t_{1/2}=0.693/(−gradient)=57.258 min; Intrisinc Clearance (CLint)=V*0.693/t_{1/2}=V*(−gradient)=24.2 μL/min/mg protein. FIG. 10 shows microsomal stability of propranolol; t_{1/2}=0.693/(−gradient)=3.56 min. Intrisinc Clearance (CLint)=V*0.693/t½=V*(−gradient)=389.6 μL/min/mg protein.

### Example 9—Solution Stability

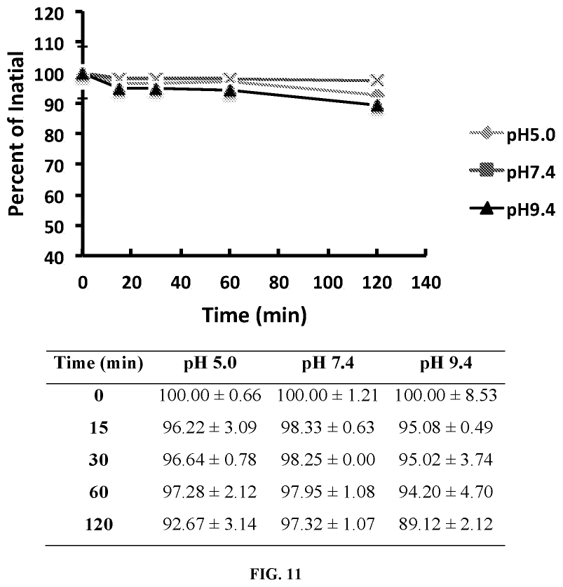

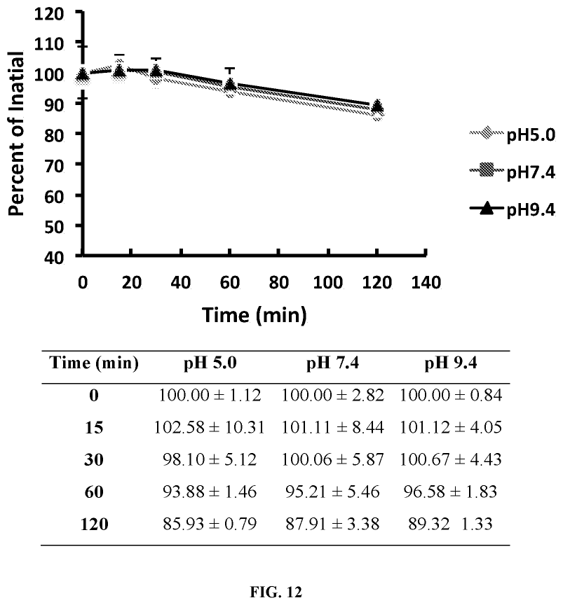

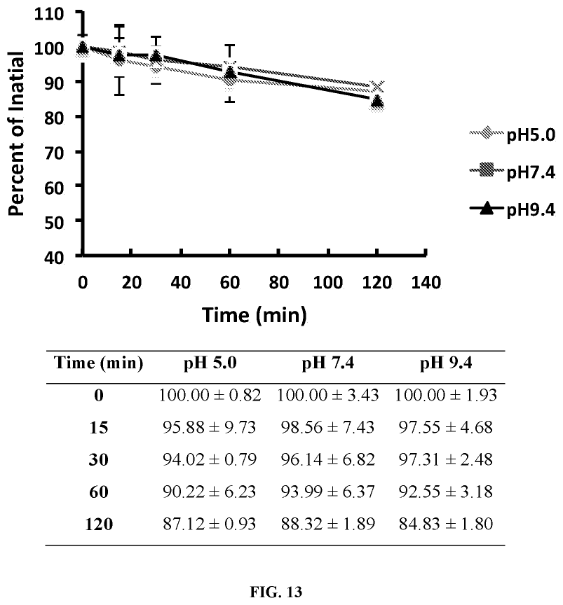

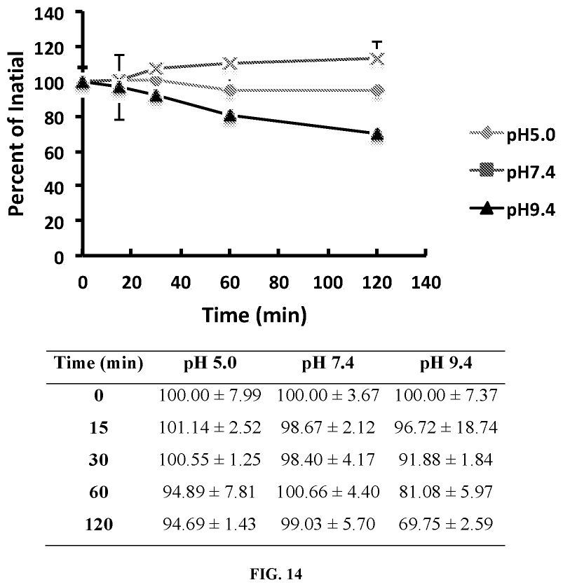

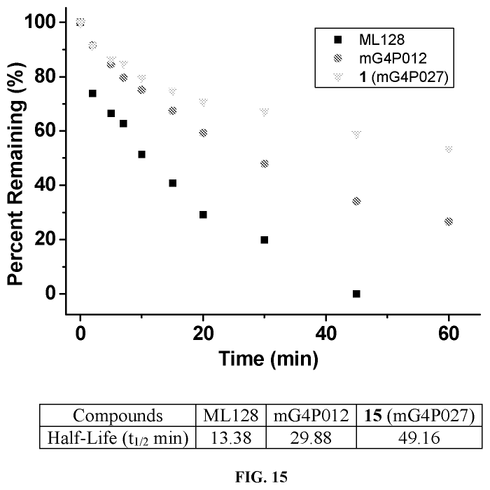

Three pH values, 5, 7.4 and 9.4 were selected to check the solution stability. Aqueous solutions with different pH values were established using the following buffers: sodium acetate-KCl—HCl buffer (20 mM, pH 5.0), phosphate buffer (20 mM, pH 7.4), and boric acid-KCl—NaOH buffer (20 mM, pH 9.4). 891 μL of each pH buffer was placed in different tubes. 9 μL (1 mM) of the sample stock (4, 13, 15 and Diltiazem) was transferred to a particular buffer tube and mixed just before the time 0 min. The mixture was vortexed and incubated at 37° C. After the incubation at the different time points (0, 15, 30, 60, and 120 min), aliquots of 50 μL samples were quenched with 100 μL of ice-cold acetonitrile with 25 μM internal reference, respectively, and analyzed by HPLC. The percentage of the remaining intact compound was calculated by (peak area at the specific time point)/(peak area at 0 min)×100. Every sample was assayed three times. See FIGS. 11-14.

### Summary of Examples 7-9

After iv injection PET tracers encounter plasma decomposition by hydrolytic enzymes in the blood and are carried into the liver where they face diverse hepatic metabolic reactions such as phase I oxidations by CYP and flavin monooxygenases (FMO). Unstable compounds often have high clearance (Clint) and short half-life (t_{1/2}) resulting in poor in vivo pharmacokinetics (PK) and unsatisfactory pharmacological performance. The in vitro plasma and liver microsomal stability of 4, 13 and 15 were studied by incubating the compounds in rat serum and rat liver microsomes as well as NADPH cofactor, respectively, using previously published methods. (refs. 45-46) Diltiazem and propranolol were used as co-assay QC controls for plasma and microsomal stability assays, respectively, to ensure that the assays were operating properly, and that the activity of the plasma and microsomes were consistent with established criteria. As Table 4 shows, 4, 13 and 15 are more stable than diltiazem in rat plasma. The results also show that 15 exhibits excellent microsomal stability and is more stable than 4 and 13, in which the suitable hepatic clearance was predicted. The solution stability of 15 was evaluated at pH 5.0, 7.4 and 9.4, respectively (ref 47). The results indicate that 15 is relatively stable in pH ranging from 5.0 to 9.4 (Table 4).

TABLE 4
Metabolic and solution stabilities.

| | | | | The solution stability | | |
|---|---|---|---|---|---|---|
| | The plasma stability | The microsome stability | | The intact ± SEM | | |
| | The intact ± SEM | t_{1/2} | Clint (μL/min/ | at 120 min (%) | | |
| Compound | at 120 min (%) | (min) | mg protein) | pH = 5.0 | pH = 7.4 | pH = 9.4 |
| 4 | 98.2 ± 6.3 | 14.4 | 96.2 | 92.7 ± 3.1 | 97.3 ± 1.1 | 89.1 ± 2.1 |
| 13 | 91.5 ± 2.5 | 25.3 | 54.8 | 85.9 ± 0.8 | 87.9 ± 3.4 | 89.3 ± 1.3 |
| 15 | 84.5 ± 10.2 | 57.3 | 24.2 | 87.1 ± 0.9 | 88.3 ± 1.9 | 84.3 ± 1.8 |
| Diltiazem | 49.0 ± 10.6 | — | — | 94.7 ± 1.4 | 99.0 ± 5.7 | 69.8 ± 2.6 |
| Propranolol | — | 3.6 | 390 | — | — | — |

The in vitro pharmacological studies reveal that compound 15 has many CNS drug-like properties, including appropriate mGluR4 affinity, potent mGluR4 PAM activity and selectivity against other mGluRs, and suitable lipophilicity as well as PPB, adequate metabolic stability and solution stability.

### Example 10—Radiosynthesis of Compound [¹⁸F]15

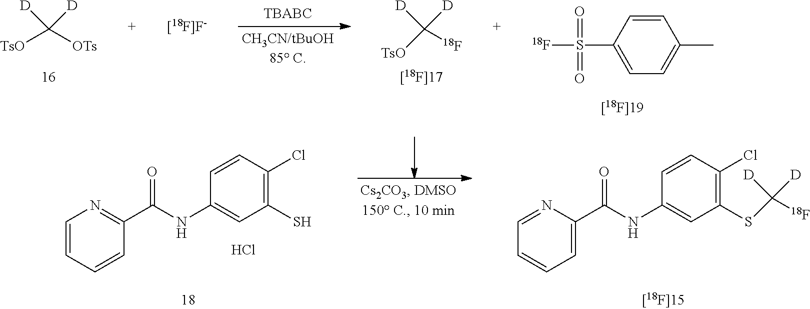

[¹⁸F]Fluoride was generated by a Siemens Eclipse HP 11 MeV cyclotron (Malvern, Pa.) using ¹⁸O-enriched water (Isoflex Isotope, San Francisco, Calif.) with proton bombardment. Fluorine-18 labeling of [¹⁸F]15 was accomplished in two steps. First, [¹⁸F]fluoride in ¹⁸O-enriched water was passed through a QMA Sep-Pak Cartridge (Waters, Milford, Mass.) to trap [¹⁸F]fluoride ions, which was washed off by 0.5 mL of the aqueous solution of tetrabutylammonium hydrogen carbonate (75 mM, from ABX advanced biochemical compounds). Acetonitrile (1.0 mL) was added to the solution and the solvents were evaporated at 115° C. in a stream of nitrogen. In order to remove water completely, 1.0 mL of acetonitrile was added and evaporated in a stream of nitrogen three more times. To the residue containing [¹⁸F]fluoride was added 16 (12.0 mg) in 0.7 mL of acetonitrile and t-BuOH (4:1). The resulting solution was heated to 85° C. for 10 min and then cooled to room temperature followed by addition of 1.5 mL of water. The mixture was then purified by a semi-preparative HPLC (Waters 4000 system equipped with an Xbridge BEH C₁₈ OBD column: 130 Å, 5 μm, 10×250 mm) by eluting with a solution of water and acetonitrile (50:50) at a flow rate of 4 mL/min to give the fractions containing [¹⁸F]17. The combined fraction was diluted with water to 40 mL and loaded on a C₁₈ Sep-Pak column. The column was further dried through a stream of nitrogen for 20-30 min. [¹⁸F]17 was washed off the Cis Sep-Pak column by 0.7 mL of dry DMSO to a reaction vessel containing 2.0 mg of 18 and cesium carbonate (3.0-5.0 mg). The resulting mixture was heated to 150° C. for 10 min and then cooled to rt, followed by addition of the HPLC eluents (2.5 mL, 0.1% formic acid solution of water and acetonitrile 40:60). The mixture was then purified using the semi-preparative HPLC (Waters 4000 system equipped with an Xbridge BEH C₁₈ OBD column: 130 Å, 5 μm, 10×250 mm) by eluting with a 0.1% formic acid solution of water and acetonitrile (40:60) at a flow rate of 4 mL/min. The fraction containing [¹⁸F]15 was diluted with water to 40 mL and loaded on a C₁₈ Sep-Pak column to give the final formulation of [¹⁸F]15 in saline containing 10% ethanol with 11.6%±2.9% radiochemical yield (RCY, n=7, decay corrected). The purity of [¹⁸F]15 was over 99% that was analyzed by an analytical HPLC (Waters 2487 series equipped with a UV detector and a BIOSCAN radioactivity detector and an ACE 5 C18-AR column: 250×10 mm, 5 m). Identity of [¹⁸F]15 was confirmed by co-injection of the cold compound 15 in HPLC analysis. The total synthesis of [¹⁸F]15 took 2.5-3.5 h with the specific activity 84.1±11.8 MBq/mmol (n=7).

### Example 11—Ex Vivo Biodistribution Studies

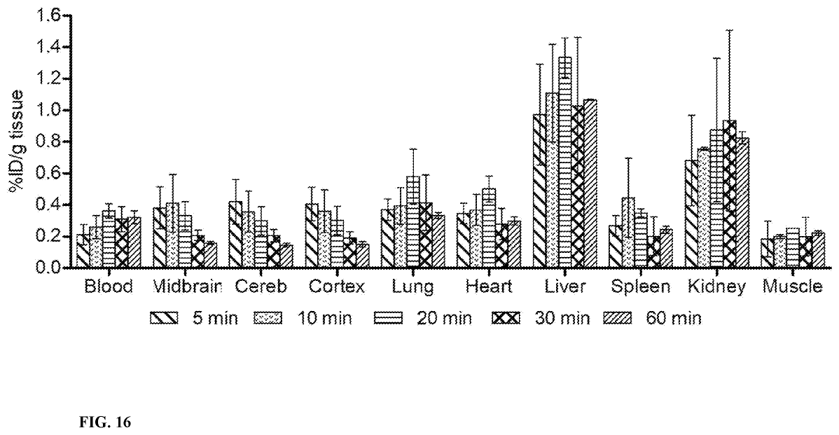

Ex vivo biodistribution studies were carried out in normal male Sprague Dawley rats to quantify accumulation of the tracer in different organs, as well as determine metabolic pathways. These studies showed reversible binding of [¹⁸F]15 in all investigated tissues with maximum uptake between 10 and 20 min except in the kidneys, where excretion of urine can affect tissue activity samples. Liver had the highest accumulation of 1.33±0.12% ID/g at 20 min followed by kidneys 0.93±0.57% ID/g at 30 min, lungs 0.58±0.17% ID/g at 20 min, heart 0.50±0.08% ID/g at 20 min and brain 0.41±0.18% ID/g at 10 min. (FIG. 16).

### Example 12—PET Imaging

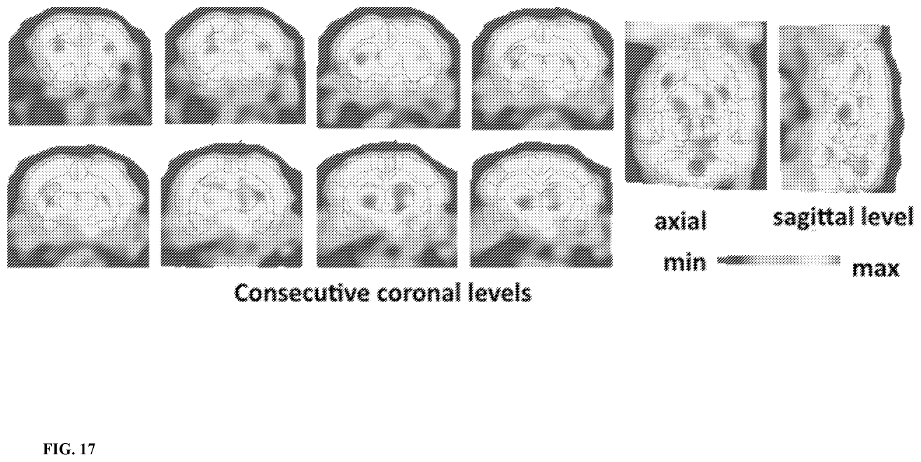

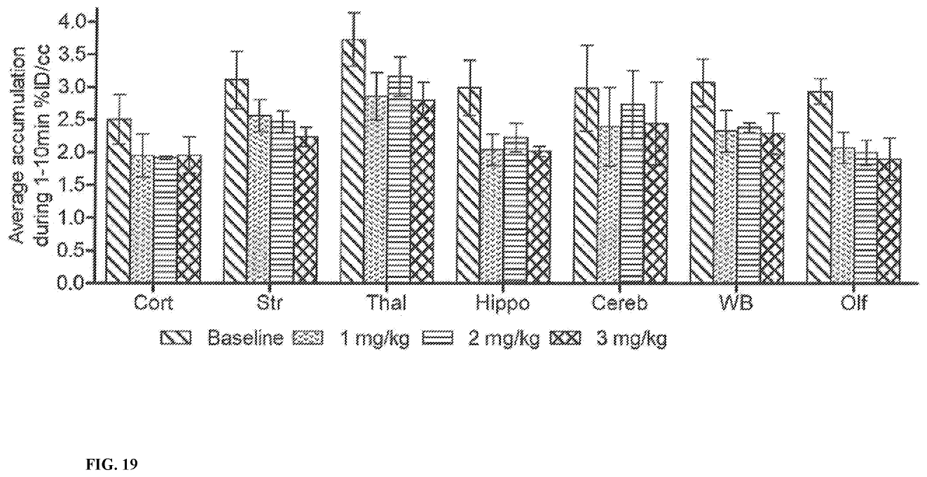

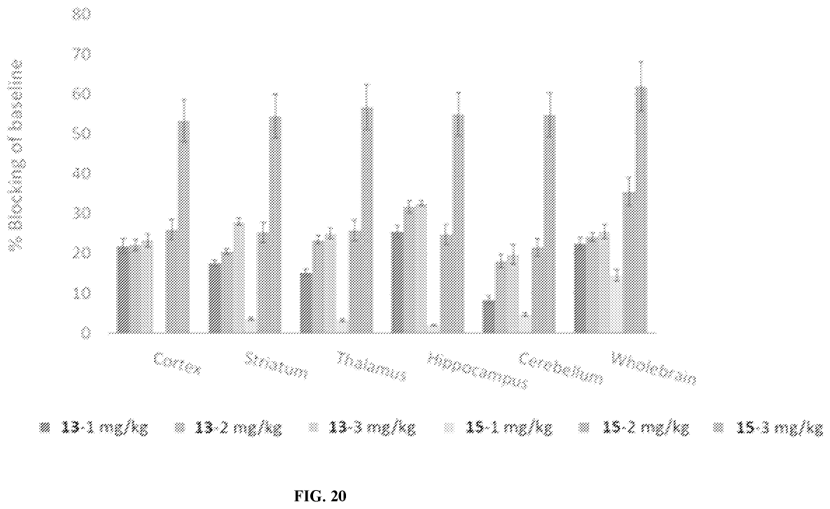

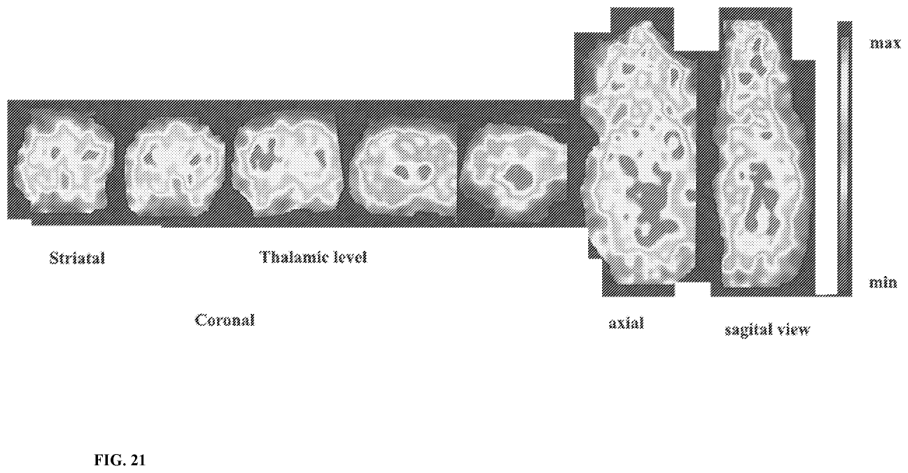

For in vivo characterization with PET imaging, [¹⁸F]15 was synthesized nine times and 30 studies were conducted in male Sprague-Dawley rats comprising 15 baseline studies and 9 blocking studies by using 13 as a blocking agent with doses of 1, 2 or 3 mg/kg and 6 self-blocking studies by using “cold” 15 as blocking agent with the same doses as 13. These studies demonstrate that [¹⁸F]15 crosses BBB and accumulates in the brain areas known to express mGluR4 (FIGS. 17 and 21). Time-activity curves (TACs) show fast reversible binding in the striatum, thalamus, cerebellum and cortex (FIG. 19). The maximum average uptake at the time interval of 1-10 min after administering of the ligand was in the thalamus, 1.77±0.26% ID/cc. The binding of [¹⁸F]15 in the rat brain was blocked in the whole brain (22-25%) by using 13 and even dose dependently in specific brain areas like striatum, 18, 20 and 28% using doses of 1, 2 or 3 mg/kg. Self-blocking with the same doses of 15 had similar blocking effect with the dose of 2 mg/kg but was significantly lower with 1 mg/kg and higher with 3 mg/kg compared to blocking with 13 (FIG. 20).

FIG. 17 shows eight consecutive coronal slices and midbrain axial and sagittal slices fused with the anatomical borderlines of different brain areas, showing the distribution of [¹⁸F]15 in the rat brain (Sprague Dawley).

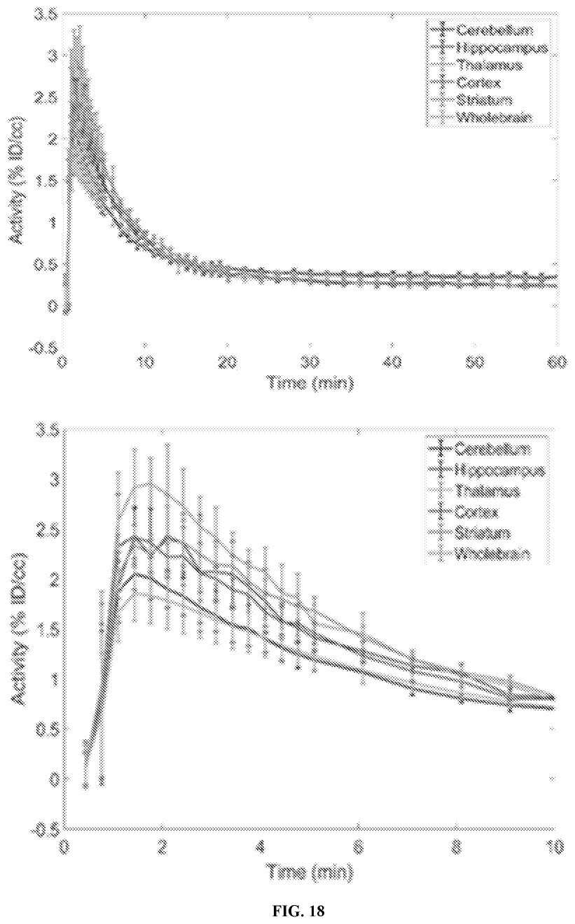

FIG. 18 shows time-activity curves showing fast accumulation and washout in all investigated brain areas. The highest accumulation was observed in the thalamus. The baseline data is averaged from five normal male rats (Sprague Dawley) with weight between 205-225 g. (left) The follow up period of 60 min shows that after 20 min the washout from striatum is low while the cerebellar activity stays nearly constant. (right) Accumulation in the different brain parts can be seen separately during the first 10 min. This image shows the highest accumulation in the thalamus achieved about 2 min after administration of the ligand.

FIG. 20 shows that blocking was calculated as a percent change from the baseline values. Blocking studies show that both 13 and 15 inhibit [¹⁸F]15 binding dose dependently. The blocking effect using 13 with the doses of 1, 2 or 3 mg/kg was from 8 to 32 percent in different brain areas while using 15 it was from 1 to 61 percent from the baseline values. However, the blocking effect with the dose of 2 mg/kg 15 was at the same level as with 13.

In sum, biodistribution studies in rats showed reversible binding of [¹⁸F]15 in all investigated tissues with maximum uptake between 10 and 20 min except in the kidneys, where excretion of urine can effect on the activity of the tissue sample. Liver had the highest accumulation of [¹⁸F]15 followed by kidney, lung, heart and brain. Time dependent accumulation of [¹⁸F]15 in the brain supports also the data obtained from the dynamic PET imaging studies of [¹⁸F]15.

PET imaging studies of regional distribution of the radioligand, [¹⁸F]15 in normal Sprague Daley rats showed the highest accumulation in the thalamus followed by striatum and hippocampus. These are the brain areas known to have the highest expression of mGlu4 receptors. The PET images illustrate the accumulated data between 2 and 10 min after administration of [¹⁸F]15. The axial and sagittal view show the activity distribution at the mid brain level. The slice thickness is 1.25 mm.

The specific binding of [¹⁸F]15 to mGlu4 receptors was investigated in normal rat brain by PET imaging studies by using pre-administration of “cold” compound, 13, with different doses. These studies show about 25-30% blocking effect by using 1 mg/kg and the blocking effect increases in several brain areas dose dependently. Large variation of the blocking effect is partially caused by the variation of the imaging time affecting the specific activity of [¹⁸F]15.

In conclusion, the results presented herein show that [¹⁸F]15 is superior imaging ligand for mGluR4 compared to previously developed and published tracers for mGluR4 (e.g., [¹⁸F]12 and [¹¹C]13).

### REFERENCES

1. Dauer, W.; et al., Parkinson's disease: Mechanisms and models. Neuron 2003, 39 (6), 889-909.
2. de Rijk, M. C.; et al., Prevalence of Parkinson's disease in Europe: A collaborative study of population-based cohorts. Neurologic Diseases in the Elderly Research Group. Neurology 2000, 54 (11 Suppl 5), S21-3.
3. Masilamoni, G. J.; et al., Metabotropic glutamate receptors: targets for neuroprotective therapies in Parkinson disease. Current Opinion in Pharmacology 2018, 38, 72-80.
4. DeLong Mahlon, R.; et al., Basal Ganglia Circuits as Targets for Neuromodulation in Parkinson Disease. JAMA neurology 2015, 72 (11), 1354-60.
5. Sebastianutto, I.; et al., mGlu receptors in the treatment of Parkinson's disease and L-DOPA-induced dyskinesia. Current Opinion in Pharmacology 2018, 38, 81-89.
6. Charvin, D., mGlu4 allosteric modulation for treating Parkinson's disease. Neuropharmacology 2018, 135, 308-315.
7. Mellone, M.; et al., Glutamatergic mechanisms in L-DOPA-induced dyskinesia and therapeutic implications. Journal of Neural Transmission 2018, 125 (8), 1225-1236.
8. Amalric, M.; et al., Group III and subtype 4 metabotropic glutamate receptor agonists: Discovery and pathophysiological applications in Parkinson's disease. Neuropharmacology 2013, 66, 53-64.
9. Huang, X.; et al., Chemical Biology of mGlu4 Receptor Activation: Dogmas, Challenges, Strategies and Opportunities. Current Topics in Medicinal Chemistry (Sharjah, United Arab Emirates) 2014, 14 (15), 1755-1770.
10. Conn, P. J.; et al., Pharmacology and functions of metabotropic glutamate receptors. Annual Review of Pharmacology and Toxicology 1997, 37, 205-237.
11. Corti, C.; et al., Distribution and synaptic localisation of the metabotropic glutamate receptor 4 (mGluR4) in the rodent CNS. Neuroscience (Oxford, United Kingdom) 2002, 110 (3), 403-420.
12. Valenti, O. M., et al., Group III metabotropic glutamate-receptor-mediated modulation of excitatory transmission in rodent substantia nigra pars compacta dopamine neurons. Journal of Pharmacology and Experimental Therapeutics 2005, 313, 1296-1304.
13. Niswender, C. M.; et al., Metabotropic glutamate receptors: physiology, pharmacology, and disease. Annual Review of Pharmacology and Toxicology 2010, 50, 295-322.
14. Goudet, C.; et al., A novel selective metabotropic glutamate receptor 4 agonist reveals new possibilities for developing subtype selective ligands with therapeutic potential. FASEB journal: official publication of the Federation of American Societies for Experimental Biology 2012, 26 (4), 1682-93.
15. Cajina, M.; et al., Qualification of LSP1-2111 as a Brain Penetrant Group III Metabotropic Glutamate Receptor Orthosteric Agonist. ACS Medicinal Chemistry Letters 2014, 5 (2), 119-123.
16. Lindsley, C. W.; et al., Metabotropic glutamate receptor 4 (mGlu4)-positive allosteric modulators for the treatment of Parkinson's disease: historical perspective and review of the patent literature. Expert Opinion on Therapeutic Patents 2012, 22 (5), 461-481.
17. Robichaud, A. J.; et al., Recent progress on the identification of metabotropic glutamate 4 receptor ligands and their potential utility as CNS therapeutics. ACS Chemical Neuroscience 2011, 17, 433-449.
18. Marino, M. J.; et al., Allosteric modulation of group III metabotropic glutamate receptor 4: A potential approach to Parkinson's disease treatment. Proceedings of the National Academy of Sciences of the United States of America 2003, 100 (23), 13668-13673.
19. Battaglia, G.; et al., Pharmacological activation of mGlu4 metabotropic glutamate receptors reduces nigrostriatal degeneration in mice treated with 1-methyl-4-phenyl-1,2,3,6-tetrahydropyridine. Journal of Neuroscience 2006, 26 (27), 7222-7229.
20. Charvin, D.; et al., Discovery, Structure-Activity Relationship, and Antiparkinsonian Effect of a Potent and Brain-Penetrant Chemical Series of Positive Allosteric Modulators of Metabotropic Glutamate Receptor 4. Journal of Medicinal Chemistry 2017, 60 (20), 8515-8537.
21. Dube, A.; et al., Therapeutic potential of metabotropic glutamate receptor 4-positive allosteric modulator TAS-4 in rodent models of movement disorders. Journal of the Neurological Sciences 2014, Ahead of Print.
22. Le Poul, E.; et al., A potent and selective metabotropic glutamate receptor 4 positive allosteric modulator improves movement in rodent models of Parkinson's disease. Journal of Pharmacology and Experimental Therapeutics 2012, 343 (1), 167-177.
23. Jones, C. K.; et al., The metabotropic glutamate receptor 4-positive allosteric modulator VU0364770 produces efficacy alone and in combination with L-DOPA or an adenosine 2A antagonist in preclinical rodent models of Parkinson's disease. Journal of Pharmacology and Experimental Therapeutics 2012, 340 (2), 404-421.
24. Niswender, C. M.; et al., Discovery, characterization, and antiparkinsonian effect of novel positive allosteric modulators of metabotropic glutamate receptor 4. Molecular Pharmacology 2008, 74 (5), 1345-1358.
25. Niswender, C. M.; et al., Development and Antiparkinsonian Activity of VU0418506, a Selective Positive Allosteric Modulator of Metabotropic Glutamate Receptor 4 Homomers without Activity at mGlu2/4 Heteromers. ACS Chem. Neurosci. 2016, 7 (9), 1201-1211.
26. Bennouar, K.-E.; et al., Synergy between L-DOPA and a novel positive allosteric modulator of metabotropic glutamate receptor 4: Implications for Parkinson's disease treatment and dyskinesia. Neuropharmacology 2013, 66, 158-169.
27. Broadstock, M.; et al., Antiparkinsonian potential of targeting group III metabotropic glutamate receptor subtypes in the rodent substantia nigra pars reticulata. British journal of pharmacology 2012, 165 (4b), 1034-45.
28. Betts, M. J.; et al., Allosteric modulation of the group III mGlu4 receptor provides functional neuroprotection in the 6-hydroxydopamine rat model of Parkinson's disease. Br. J. Pharmacol. 2012, 166 (8), 2317-2330.
29. Ponnazhagan, R.; et al., The Metabotropic Glutamate Receptor 4 Positive Allosteric Modulator ADX88178 Inhibits Inflammatory Responses in Primary Microglia. Journal of neuroimmune pharmacology 2016, 11 (2), 231-7.
30. Hong, S.-P.; et al., Tricyclic Thiazolopyrazole Derivatives as Metabotropic Glutamate Receptor 4 Positive Allosteric Modulators. Journal of Medicinal Chemistry 2011, 54 (14), 5070-5081.
31. Zhang, Z.; et al., Imaging of Metabotropic Glutamate Receptors (mGluR)s. In Neuroimaging—Clinical applications Bright, P., Ed. InTech—Open Access Publisher: Rijeka, Croatia, 2012; pp 499-532.
32. Kil, K.-E; et al., Radiosynthesis of N-(4-chloro-3-[¹¹C]methoxyphenyl)-2-picolinamide ([¹¹C]ML128) as a PET radiotracer for metabotropic glutamate receptor subtype 4 (mGlu₄). Bioorganic and Medicinal Chemistry 2013, 21, 5955-5962.
33. Kil, K.-E.; et al., Radiosynthesis and Evaluation of an 18F-Labeled Positron Emission Tomography (PET) Radioligand for Metabotropic Glutamate Receptor Subtype 4 (mGlu4). Journal of Medicinal Chemistry 2014, 57 (21), 9130-9138.
34. Kil, K.-E.; et al., Synthesis and evaluation of N-(methylthiophenyl)picolinamide derivatives as PET radioligands for metabotropic glutamate receptor subtype 4. Bioorganic & Medicinal Chemistry Letters 2016, 26 (1), 133-139.
35. Fujinaga, M.; et al., Radiosynthesis and evaluation of 5-methyl-N-(4-[11C]methylpyrimidin-2-yl)-4-(1H-pyrazol-4-yl)thiazol-2-amine ([11C]ADX88178) as a novel radioligand for imaging of metabotropic glutamate receptor subtype 4 (mGluR4). Bioorganic & Medicinal Chemistry Letters 2016, 26 (2), 370-374.
36. Takano, A.; et al., Characterization of [11C]PXT012253 as a PET Radioligand for mGlu4 Allosteric Modulators in Nonhuman Primates. Molecular Imaging and Biology 2018, Ahead of Print.
37. Charvin, D.; et al., An mGlu4-Positive Allosteric Modulator Alleviates Parkinsonism in Primates. Movement Disorders 2018, 33 (10), 1619-1631.
38. Zhang, Z.; et al., Re-exploring the N-phenylpicolinamide derivatives to develop mGlu4 ligands with improved affinity and in vitro microsomal stability. Bioorganic & Medicinal Chemistry Letters 2015, 25 (18), 3956-3960.
39. Wang, J.; et al., Improved synthesis of the thiophenol precursor N-(4-chloro-3-thiophenyl)picolinamide for making the mGluR4 PET ligand. Tetrahedron 2019.
40. Poutiainen, P.; et al., Co-operative binding assay for the characterization of mGlu4 allosteric modulators. Neuropharmacology 2015, 97, 7.
41. Roth, B. L., Assay protocol book. Version III ed.; PDSP, N., Ed. Department of Pharmacology, University of North Carolina at Chapel Hill, 2018.
42. DiRaddo, J. O.; et al., A real-time method for measuring cAMP production modulated by Gαi/o-coupled metabotropic glutamate receptors. Journal of Pharmacology and Experimental Therapeutics 2014, 349 (3), 373-382, 10 pp.
43. Pike, V. W., PET radiotracers: crossing the blood-brain barrier and surviving metabolism. Trends in Pharmacological Sciences 2009, 30 (8), 431-440.
44. Honer, M.; et al., Radioligand development for molecular imaging of the central nervous system with positron emission tomography. Drug Discovery Today 2014, 19 (12), 1936-1944.
45. Di, L.; et al., Development and application of high throughput plasma stability assay for drug discovery. International Journal of Pharmaceutics 2005, 297 (1), 110-119.
46. Houston, J. B., Utility of in vitro drug metabolism data in predicting in vivo metabolic clearance. Biochemical Pharmacology 1994, 47 (9), 1469-79.
47. Di, L.; et al., Development and application of an automated solution stability assay for drug discovery. Journal of Biomolecular Screening 2006, 11 (1), 40-47.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A compound of Formula (I): 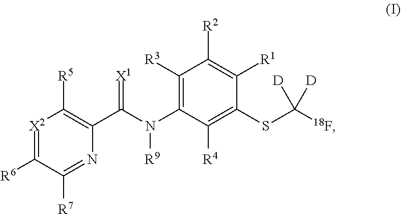
or a pharmaceutically acceptable salt thereof, wherein:
R¹ is selected from halo, CN, NO₂, C₁₋₆ alkylthio, C₁₋₃ haloalkyl, C₁₋₃ haloalkoxy, 4-10 membered heterocycloalkyl, NHC(O)Cy¹, NHS(O)₂Cy¹, C(O)NHCy¹, and S(O)₂NHCy¹; wherein said 4-10 membered heterocycloalkyl is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy;
each Cy¹ is independently an C₆₋₁₀ aryl, optionally substituted 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy;
R², R³, and R⁴ are each independently selected from H, OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, amino, C₁₋₆ alkylamino, di(C₁₋₆ alkyl)amino, thio, and C₁₋₆ alkylthio;
R⁹ is selected from H and C₁₋₃ alkyl;
X² is selected from N and CR⁸;
R⁵, R⁶, R⁷, and R⁸ are each independently selected from H, OH, NO₂, CN, halo, C₁₋₃ alkyl, C₁₋₃ alkoxy, C₁₋₃ haloalkyl, C₁₋₃ haloalkoxy, cyano-C₁₋₃ alkyl, HO—C₁₋₃ alkyl, amino, C₁₋₆ alkylamino, di(C₁₋₆ alkyl)amino, thio, and C₁₋₆ alkylthio;
X¹ is selected from O, S, and NR^{e1}; and
R^{e1} is selected from H, C₁₋₄ alkyl, C₁₋₄ alkoxy, OH, and CN;
or R⁵ and R^{e1} together with the N atom to which R^{e1} is attached and the carbon atom to which R⁵ is attached for a 5-10 membered heterocycloalkyl ring, which is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy.

2. The compound of claim 1, wherein:
R¹ is selected from halo, NO₂, C₁₋₆ alkylthio, 4-10 membered heterocycloalkyl, NHC(O)Cy¹, and S(O)₂NHCy¹; wherein said 4-10 membered heterocycloalkyl is optionally substituted with halo or C₁₋₃ alkyl;
each Cy¹ is independently C₆₋₁₀ aryl, optionally substituted with halo or C₁₋₃ alkyl;
R², R³, and R⁴ are each independently selected from H, NO₂, halo, C₁₋₃ alkyl, C₁₋₃ alkoxy, C₁₋₃ alkylthio, and C₁₋₃ haloalkyl;
R⁹ is H; and
R⁵, R⁶, R⁷, and R⁸ are each independently selected from H, OH, amino, halo, C₁₋₃ alkyl, C₁₋₃ alkoxy, C₁₋₃ haloalkyl, thio, and C₁₋₆ alkylthio.

3. The compound of claim 1, wherein the compound of Formula (I) has formula: 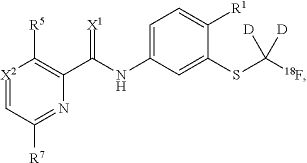
or a pharmaceutically acceptable salt thereof, wherein:
R¹ is selected from halo and NO₂;
X¹ is selected from O and S;
X² is selected from N and CH;
R⁵ is selected from H, amino, and OH; and
R⁷ is selected from H and halo.

4. The compound of claim 3, wherein the compound has formula: 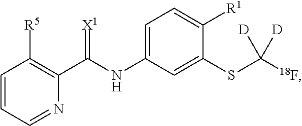
or a pharmaceutically acceptable salt thereof, wherein:
R⁵ is selected from H and amino.

5. The compound of claim 1, wherein the compound of Formula (I) has formula: 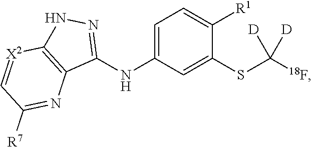
or a pharmaceutically acceptable salt thereof, wherein:
R¹ is selected from halo and NO₂;
X² is selected from N and CH; and
R⁷ is selected from H and halo.

6. The compound of claim 1, wherein the compound of Formula (I) is selected from any one of the following compounds, or a pharmaceutically acceptable salt thereof:

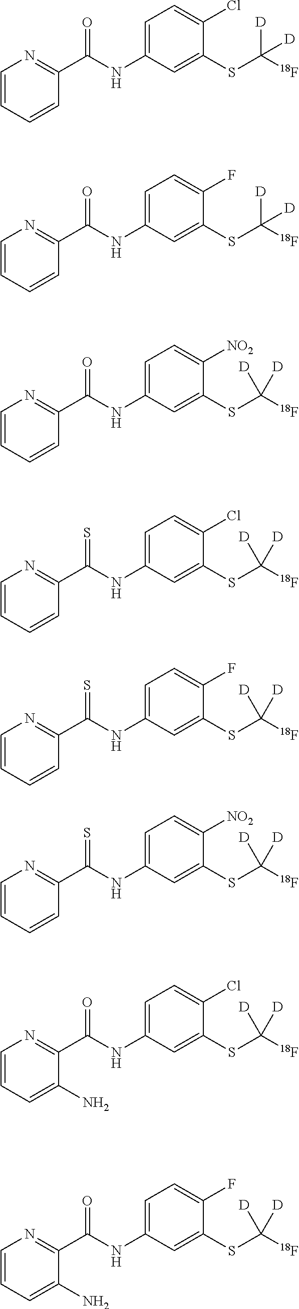

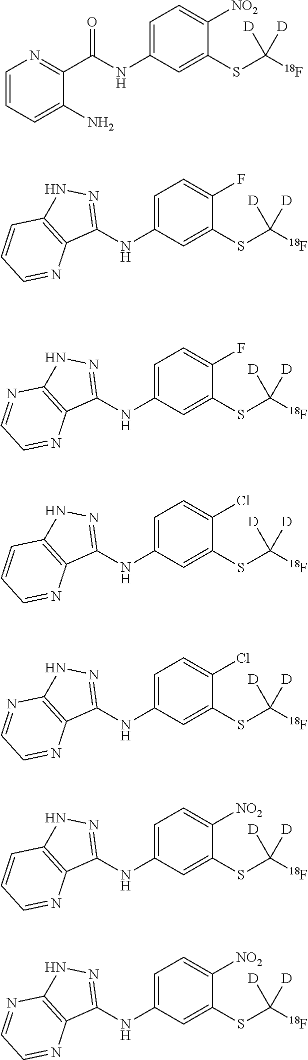

7. A pharmaceutical composition comprising a compound of claim 1, or a pharmaceutically acceptable salt thereof, and a pharmaceutically acceptable carrier.

8. A method of imaging a brain of a subject, the method comprising:
i) administering to the subject an effective amount of a compound of claim 1, or a pharmaceutically acceptable salt thereof;
ii) waiting a time sufficient to allow the compound to accumulate in the brain to be imaged; and
iii) imaging the brain with an imaging technique.

9. A method of monitoring treatment of a neurological disorder associated with mGluR4 in a subject, the method comprising:
i) administering to the subject an effective amount of a compound of claim 1, or a pharmaceutically acceptable salt thereof;
ii) waiting a time sufficient to allow the compound of claim 1 to accumulate in a brain of the subject;
iii) imaging the brain of the subject with an imaging technique;
iv) administering to the subject a therapeutic agent in an effective amount to treat the neurological disorder;
v) after iv), administering to the subject an effective amount of a compound of claim 1, or a pharmaceutically acceptable salt thereof;
vi) waiting a time sufficient to allow the compound of claim 1 to accumulate in the brain of the subject;
vii) imaging the brain of the subject with an imaging technique; and
viii) comparing the image of step iii) and the image of step vii).

10. The method of claim 8, wherein the imaging technique is selected from positron emission tomography (PET) imaging, positron emission tomography with computer tomography (PET/CT) imaging, and positron emission tomography with magnetic resonance (PET/MRI) imaging.

11. The method of claim 9, wherein the neurological disorder associated with mGluR4 is selected from Parkinson's disease, dyskinesia, Lewy body disease, Prion disease, motor neuron disease (MND), and Huntington's disease.

12. A compound of Formula (II): 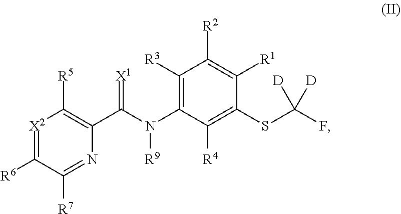
or a pharmaceutically acceptable salt thereof, wherein:
R¹ is selected from halo, CN, NO₂, C₁₋₆ alkylthio, C₁₋₃ haloalkyl, C₁₋₃ haloalkoxy, 4-10 membered heterocycloalkyl, NHC(O)Cy¹, NHS(O)₂Cy¹, C(O)NHCy¹, and S(O)₂NHCy¹;
wherein said 4-10 membered heterocycloalkyl is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy;
each Cy¹ is independently an C₆₋₁₀ aryl, optionally substituted 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy;
R², R³, and R⁴ are each independently selected from H, OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, amino, C₁₋₆ alkylamino, di(C₁₋₆ alkyl)amino, thio, and C₁₋₆ alkylthio;
R⁹ is selected from H and C₁₋₃ alkyl;
X² is selected from N and CR⁸;
R⁵, R⁶, R⁷, and R⁸ are each independently selected from H, OH, NO₂, CN, halo, C₁₋₃ alkyl, C₁₋₃ alkoxy, C₁₋₃ haloalkyl, C₁₋₃ haloalkoxy, cyano-C₁₋₃ alkyl, HO—C₁₋₃ alkyl, amino, C₁₋₆ alkylamino, di(C₁₋₆ alkyl)amino, thio, and C₁₋₆ alkylthio;
X¹ is selected from O, S, and NR^{e1}; and
R^{e1} is selected from H, C₁₋₄ alkyl, C₁₋₄ alkoxy, OH, and CN;
or R⁵ and R^{e1} together with the N atom to which R^{e1} is attached and the carbon atom to which R⁵ is attached for a 5-10 membered heterocycloalkyl ring, which is optionally substituted with 1, 2, or 3 substituents independently selected from OH, NO₂, CN, halo, C₁₋₃ alkyl, C₁₋₃ alkoxy, C₁₋₃ haloalkyl, and C₁₋₃ haloalkoxy.

13. The compound of claim 12, wherein:
R¹ is selected from halo, NO₂, C₁₋₆ alkylthio, 4-10 membered heterocycloalkyl, NHC(O)Cy¹, and S(O)₂NHCy¹; wherein said 4-10 membered heterocycloalkyl is optionally substituted with halo or C₁₋₃ alkyl;
each Cy¹ is independently C₆₋₁₀ aryl, optionally substituted with halo or C₁₋₃ alkyl;
R², R³, and R⁴ are each independently selected from H, NO₂, halo, C₁₋₃ alkyl, C₁₋₃ alkoxy, C₁₋₃ alkylthio, and C₁₋₃ haloalkyl;
R⁹ is H; and
R⁵, R⁶, R⁷, and R⁸ are each independently selected from H, OH, amino, halo, C₁₋₃ alkyl, C₁₋₃ alkoxy, C₁₋₃ haloalkyl, thio, and C₁₋₆ alkylthio.

14. The compound of claim 12, wherein the compound of Formula (II) has formula: 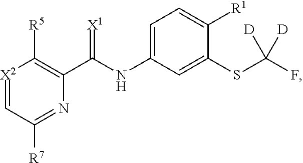
or a pharmaceutically acceptable salt thereof, wherein:
R¹ is selected from halo and NO₂;
X¹ is selected from O and S;
X² is selected from N and CH;
R⁵ is selected from H, amino, and OH; and
R⁷ is selected from H and halo.

15. The compound of claim 14, wherein the compound has formula: 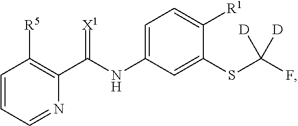
or a pharmaceutically acceptable salt thereof, wherein:
R⁵ is selected from H and amino.

16. The compound of claim 12, wherein the compound of Formula (II) has formula: 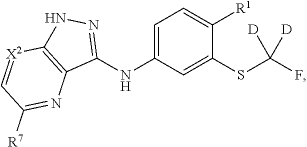
or a pharmaceutically acceptable salt thereof, wherein:
R¹ is selected from halo and NO₂;
X² is selected from N and CH; and
R⁷ is selected from H and halo.

17. The compound of claim 12, wherein the compound of Formula (II) is selected from any one of the following compounds, or a pharmaceutically acceptable salt thereof:

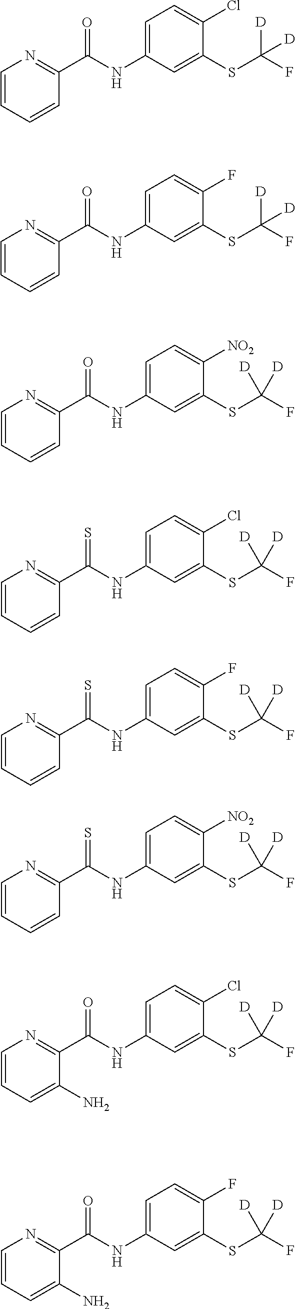

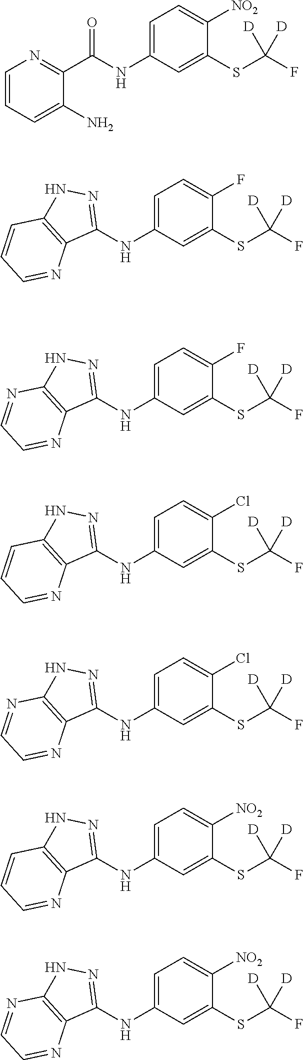

18. A pharmaceutical composition comprising a compound of claim 12, or a pharmaceutically acceptable salt thereof, and a pharmaceutically acceptable carrier.

19. A method of treating a neurological disorder associated with mGluR4 in a subject, the method comprising administering to the subject in need thereof a therapeutically effective amount of a compound of claim 12, or a pharmaceutically acceptable salt thereof.

20. The method of claim 19, wherein the neurological disorder associated with mGluR4 is selected from Parkinson's disease, dyskinesia, Lewy body disease, Prion disease, motor neuron disease (MND), and Huntington's disease.
